(19) United States
(12) Patent Application Publication
(10) Pub. No.: US 2026/0256871 A1
(43) Pub. Date: Sep. 3, 2026

(54) COMPOSITIONS AND METHODS FOR TREATMENT OF SUBJECTS SUFFERING FROM A SARS-COV-2 INFECTION

(71) Applicants: RENOVION, INC., Chapel Hill, NC (US); THE UAB RESEARCH FOUNDATION, Birmingham, AL (US)

(72) Inventors: Carolyn DURHAM, Chapel Hill, NC (US); Dan COPELAND, Chapel Hill, NC (US); Steven M. ROWE, Birmingham, AL (US); Javier CAMPOS-GOMEZ, Birmingham, AL (US)

(21) Appl. No.: 18/994,513

(22) PCT Filed: Jul. 17, 2023
(86) PCT No.: PCT/US2023/070345

§ 371 Date: Jan. 14, 2025

Related U.S. Application Data
(60) Provisional application No. 63/389,752, filed on Jul. 15, 2022.

(51) Int. Cl. A61K 38/06; A61K 31/375; A61P 11/00; A61P 31/14

(52) U.S. Cl. CPC A61K 38/063; A61K 31/375; A61P 11/00; A61P 31/14

(57) ABSTRACT

The application is directed to methods for treatment or prevention of SARS-CoV-2 infection in a subject in need thereof, the composition comprising an organic acid (e.g., ascorbic acid), glutathione, a glutathione derivative, a glutathione conjugate, a pharmaceutically acceptable salt thereof, and methods of using the same.

### CROSS REFERENCE TO RELATED APPLICATIONS

This application claims priority to U.S. Provisional Patent Application No. 63/389,752, filed Jul. 15, 2022, which is incorporated herein by reference in its entirety.

### STATEMENT OF GOVERNMENT FUNDING

This disclosure was made with government support under grants HL135816 and DK072482 awarded by the NIH. The government has certain rights in the invention.

### FIELD OF THE DISCLOSURE

The present disclosure relates to the field of microbiology and diseases of the respiratory system.

### BACKGROUND

Severe acute respiratory syndrome coronavirus 2 (SARS-CoV-2) is a coronavirus that causes coronavirus disease 2019 (COVID-19) and is responsible for the COVID-19 pandemic. A wide range of symptoms have been reported for patients suffering from COVID-19, ranging from mild symptoms to severe illness, which can lead to death. Common symptoms include: fever, respiratory symptoms, dyspnea, cough, fatigue, aches, loss of taste and/or smell, and intestinal distress (e.g., nausea, vomiting, and/or diarrhea).

SARS-CoV-2 infection can result in severe pulmonary inflammation as part of the disease known as coronavirus disease, COVID or COVID-19. A notable characteristic of COVID is destruction of the ciliated cells of the respiratory epithelium, which disrupts the protection of the mucociliary transport (MCT) apparatus, an innate defense of the lung (Robinot, R. et al., Nature Communications 2021 12:1 12, 1-16 (2021); Bridges, J. P., Vladar, E. K., Huang, H. & Mason, R. J., Thorax 77, 203-209 (2022); and Zhu, N. et al., Nat. Commun. 11, (2020)). As viral particles accumulate and are transmitted within the distal airway, they contribute to mucus plugging (a prominent finding in severe cases of COVID-19), which increases the risk of secondary infections (Ripa, M. et al., Clin Microbiol Infect 27, 451-457 (2021); and Chong, W. H., Saha, B. K., Ananthakrishnan Ramani & Chopra, A., Infection 49, 591-605 (2021)).

Although vaccination has improved the incidence and severity of the disease, severe respiratory disease continues to affect a substantial portion of the population and has been exacerbated by the emergence of more contagious variants of SARS-CoV2. Antiviral treatments such as monoclonal antibodies (e.g., sotrovimab; bebtelovimab) and small molecule antivirals (e.g., remdesivir, paxlovid, molnupiravir) have reduced disease severity (Şimşek-Yavuz, S. & Komsuoǧlu Çelikyurt, F. I., Turk J Med Sci 51, 3372-3390 (2021); Parums, D. v. Editorial: Current Status of Oral Antiviral Drug Treatments for SARS-CoV-2 Infection in Non-Hospitalized Patients. Med Sci Monit 28, (2022); Bartoletti, M. et al. ESCMID COVID-19 living guidelines: drug treatment and clinical management: author's reply. Clin Microbiol Infect (2022) doi:10.1016/j.cmi.2021.12.025; Aleem, A. & Slenker, A. K., StatPearls (2021); Lamontagne, F. et al. A living WHO guideline on drugs for covid-19. The BMJ 370, (2020); and Kreuzberger, N. et al. SARS-CoV-2-neutralising monoclonal antibodies for treatment of COVID-19. Cochrane Database Syst Rev 9, CD013825 (2021)), and corticosteroid treatment have reduced mortality in individuals with severe forms of the disease (Parasher, A. COVID-19: Current understanding of its Pathophysiology, Clinical presentation and Treatment. Postgraduate Medical Journal 97, 312-320 (2021); and Gavriatopoulou, M. et al., Clin Exp Med 21, 167-179 (2021)). However, morbidity and mortality remain unacceptably high, especially for patients with one or more risk factors for severe infection. Moreover, patients with severe COVID-19 often require supplemental oxygen and mechanical ventilation, and patients with “long COVID” can suffer from to new, recurring, or ongoing symptoms and clinical findings weeks after infection with SARS-CoV-2, sometimes after recovery from initial symptoms and clinical effects (e.g., ARDS).

Despite numerous ongoing trials of antiviral and immunosuppressive agents, no current therapy addresses the underlying neutrophilic inflammation and mucus plugging that results in hypoxia and airway destruction, clinical decline, subsequent infection or pneumonia, and high risk of mortality in COVID-19 or long COVID patients. Accordingly, there is a present need to find safe, easy-to-use, and effective drugs to treat patients with viral respiratory infections such as SARS-CoV-2.

### BRIEF SUMMARY

Certain aspects of the disclosure are directed to a method of treating, preventing, or reducing the risk of an infection by or symptoms associated with SARS-CoV-2 in a subject in need thereof, comprising administering to the subject (e.g., to the subject's airway) a therapeutically effective amount of a composition comprising: (a) glutathione, a glutathione derivative, a glutathione conjugate, or a pharmaceutically acceptable salt of glutathione, a glutathione derivative, or a glutathione conjugate; and (b) an organic acid or a pharmaceutically acceptable salt thereof.

Certain aspects of the disclosure are directed to a method of protecting the lungs of a subject suffering from SARS-CoV-2 from cilia damage comprising administering to the airway of the subject a composition comprising: (a) glutathione, a glutathione derivative, a glutathione conjugate, or a pharmaceutically acceptable salt of glutathione, a glutathione derivative, or a glutathione conjugate; and (b) an organic acid or a pharmaceutically acceptable salt thereof. In some aspects, the cilia damage is cilia shortening. In some aspects, the protection comprises reducing the incidence of damage. In some aspects, the method (i) increases the ciliary beat frequency of the patient's airway epithelial cells, (ii) increases the number of the cilia in the airway of the subject, and/or (iii) increases the length of the cilia in the airway of the subject

In some aspects, the methods disclosed herein comprises administering to the subject (e.g., to the subject's airway) a therapeutically effective amount of a composition comprising: (a) glutathione or a pharmaceutically acceptable salt of glutathione; and (b) ascorbic acid or a pharmaceutically acceptable salt thereof.

In some aspects, the one or more symptoms or signs of infection by SARS-CoV-2 comprise lung inflammation, fever, chills, cough, shortness of breath, difficulty breathing, fatigue, muscle aches, body aches, headache, reduction in ability to taste, reduction in ability to smell, sore throat, congestion, runny nose, nausea, vomiting, diarrhea, chest pain, confusion, inability to wake, inability to stay awake, discolored skin, discolored lips, or discolored nail beds.

In some aspects, the molar ratio of (a):(b) is about 0.1-0.5:0.5-1.

In some aspects, the composition further comprises (c) a bicarbonate or a pharmaceutically acceptable salt thereof.

In some aspects, the bicarbonate or a pharmaceutically acceptable salt thereof comprises sodium bicarbonate or calcium bicarbonate.

In some aspects, the molar ratio of (a):(b):(c) is about 0.1-0.6:0.5-1:1; about 0.4-0.6:0.4-0.6:1; or about 0.5:0.5:1.

In some aspects, the SARS-CoV-2 is a variant is selected from the group consisting of an Alpha variant (e.g., B.1.1.7 and Q lineages), a Beta variant (e.g., B.1.351 and descendent lineages), a Gamma variant (e.g., P.1 and descendent lineages), a Delta variant (e.g., B.1.617.2 and AY lineages), an Epsilon variant (e.g., B.1.427 and B.1.429), an Eta variant (e.g., B.1.525), an Iota variant (e.g., B.1.526), a Kappa variant (e.g., B.1.617.1), 1.617.3, a Mu variant (e.g., B.1.621, B.1.621.1), a Zeta variant (e.g., P.2), or an Omicron variant (e.g., B.1.1.529, BA.1, BA.1.1, BA.2, BA.3, BA.4 and BA.5 lineages).

In some aspects, the composition comprises glutathione.

In some aspects, the organic acid is ascorbic acid or a pharmaceutically acceptable salt thereof.

In some aspects, the administration is to the airway via inhalation. In some aspects, the composition is administered to the lungs by an inhalable dosage form.

In some aspects, the inhalable dosage form is a metered dose inhaler, a dry powder inhaler, or a nebulizer.

In some aspects, the subject has long COVID.

In some aspects, the subject has developed one or more SARS-CoV-2-related sequelae. In some aspects, the one or more SARS-CoV-2-related sequelae comprises a bronchiectasis. In some aspects, the bronchiectasis is traction bronchiectasis. In some aspects, the bronchiectasis is non-cystic fibrosis bronchiectasis. In some aspects, the one or more SARS-CoV-2-related sequelae is characterized by a symptom selected from one or more of ageusia, myalgia, arthralgia, parosmia, anosmia, fatigue, headache, cough, chills, shivers, fever, dyspnea, sore throat, rhinorrhea, diarrhea, brain fog, nausea, subjective fever, abdominal pain, vomiting, rash, skin abnormality, and blood clots.

In some aspects, the subject has a chronic airway disease or condition. In some aspects, the chronic airway disease or condition is a pre-existing condition.

In some aspects, the subject belongs to a subject population having an increased risk of SARS-CoV-2 infection, or an increased risk of a severe infection by SARS-CoV-2.

In some aspects, the subject is aged 65 or greater.

In some aspects, the subject suffers from an acute SARS-CoV-2 infection.

In some aspects, the subject suffers from an acute infection by a pathogen. In some aspects, the pathogen is SARS-CoV-2.

In some aspects, the subject has a pulmonary or airway disease or disorder. In some aspects, the pulmonary or airway disease or disorder is cystic fibrosis or non-cystic fibrosis bronchiectasis. In some aspects, the subject is a solid organ transplant (e.g., a lung transplant), or a blood or bone marrow transplant recipient.

In some aspects, the composition comprises: (a) glutathione, a glutathione derivative, a glutathione conjugate, a pharmaceutically acceptable salt thereof, or any combination thereof, and (b) an organic acid, wherein the molar ratio of (a) to (b) is about 0.5-1:1 and the pH of the formulation is at least 5.5. In some aspects, the organic acid is ascorbic acid. In some aspects, the composition further comprises (c) a bicarbonate salt (e.g., sodium bicarbonate or calcium bicarbonate). In some aspects, the composition does not include a bicarbonate salt.

In some aspects, the molar ratio of (a):(b):(c) is about 0.1-0.5:0.5-1:1. In some aspects, the molar ratio of (a):(b):(c) is about 0.4-0.5:0.5-1:1. In some aspects, the molar ratio of (a):(b):(c) is about 0.4-0.5:0.5:1 or 0.4-0.5:1:1.

In some aspects, the pH of the composition is about 5.5 to about 10, about 5.5 to about 8, about 6 to about 10, or about 6 to about 8. In some aspects, the pH is about 5.5, about 6.5, about 7.0, or about 7.5. In some aspects, the pH of the composition is 7±1.5. In some aspects, the pH of the composition is about 6.

In some aspects, the composition is an aqueous solution, a dry powder, or lyophilized.

In some aspects, the present disclosure provides a method of upregulating mucociliary clearance and/or ciliation in a subject suffering from or at risk of impaired mucociliary clearance and/or ciliation comprising administering to the subject a composition disclosed herein. In some aspects, the subject has a SARS-CoV-2 infection. In some aspects, the subject has long COVID. In some aspects, the subject has inflammation incident to a SARS-CoV-2 infection. In some aspects, administering the composition decreases mucus viscosity of the patient. In some aspects, administering the composition increases ciliary beat frequency of the patient's airway epithelial cells. In some aspects, administering the composition increases the number of the cilia in the airway of the subject. In some aspects, administering the composition increases the length of the cilia in the airway of the subject. In some aspects, administering the composition increases the mucociliary transport rate of the patient's airway epithelial cells. In some aspects, administering said composition increases the airway surface liquid height of the patient.

In some aspects, the present disclosure provides a method of upregulating mucociliary clearance in a subject suffering from or at risk of SARS-CoV-2. In some aspects, the present disclosure provides a method of upregulating ciliary expression and/or function in a subject suffering from or at risk of impaired ciliary expression and/or function comprising administering to the subject a composition disclosed herein. In some aspects, the subject is suffering from long COVID. In some aspects, the subject is suffering from inflammation incident to a SARS-CoV-2 infection. In some aspects, the subject is oxygen dependent.

In some aspects, administering the composition upregulates mucus clearance without resulting in neutrophilia.

In some aspects, administration of the composition according to the methods disclosed herein blocks SARS-CoV-2 replication. In some aspects, administration of the composition according to the methods disclosed herein reduces SARS-CoV-2 viral load. In some aspects, administration of the composition according to the methods disclosed herein does not decrease SARS-CoV-2 infectivity.

### BRIEF DESCRIPTION OF THE DRAWINGS/FIGURES

FIGS. 1A-1C provide HBEC-ALI-based antiviral assay and validation. FIG. 1A provides a schematic representation of an antiviral assay. FIG. 1B provides a graph of viral copy number measured by the RT-qPCR for a representative experiment with three filter replicates per condition. Camostat was added basolaterally, and the vehicle used was DMSO, the same solvent used to dissolve the compound. FIG. 1C provides data from two independent experiments, as the one shown in FIG. 1B, were converted to percent of viral inhibition, and averaged (3 replicates/experiment, n=6 per condition,). Camostat significantly inhibited at concentrations higher than 16.62 uM compared to the vehicle (P<0.0001).

FIGS. 2A-2H show antiviral activity and toxicity for different mucoactive agents. FIG. 2A shows the effect of ivacaftor at increasing concentrations (10, 20, and 30 μM) compared to vehicle on the viral copy number measured by the RT-qPCR. FIG. 2B shows data from the graph in FIG. 2A converted to percent viral inhibition. FIGS. 2C-2D show the effect of PAAG compared to vehicle on the viral copy number and a graph of the results in percent viral inhibition, respectively. 2E-2F show the effect of HA compared to vehicle on the viral copy number and a graph of the results in percent viral inhibition, respectively. 2G-2H show the effect of ARINA-1 compared to vehicle on the viral copy number and a graph of the results in percent viral inhibition, respectively. DMSO was the vehicle used for ivacaftor (hydrophobic compound) and saline for PAAG, HA and ARINA-1 (hydrophilic compounds). All experiments were performed at least in duplicate independent assays, each with at least three transwell filter replicates per condition. Treatments were compared using ordinary one-way ANOVA statistical analysis. Hydrophobic compounds (added basolaterally) and hydrophilic (added apically) are shown in green and red, respectively. For each compound, each independent experiment was done with primary HBEC from a different donor.

FIG. 21 shows cytotoxicity of the compounds (ivacaftor, PAAG, HA, and ARINA-1) tested at the maximum concentration used in the antiviral assays (see FIGS. 2A-2H). DMSO was the vehicle used for ivacaftor and camostat mesylate (hydrophobic compounds) and saline for PAAG, HA and ARINA-1 (hydrophilic compounds). All experiments were performed at least in duplicate independent assays, each with three transwell filter replicates per condition. Treatments were compared using ordinary one-way ANOVA statistical analysis.

FIGS. 3A-3M provides histopathology studies showing that ARINA-1 protected HBECs from SARS-CoV2-mediated cytopathology. FIGS. 3A-3L provide representative photo micrographs of HBEC cross-sections with the immunohistochemistry and treatments. Each row corresponds to the immunohistochemistry using the antibody against the cell marker shown at the left, and each column corresponds to the treatment shown above the upper pictures. SARS-CoV2 caused cilia loss and shortening in saline-treated cells (FIG. 3B). However, ARINA-1 protected cilia from damage (FIG. 3D). Similarly, the virus induced significant apoptosis in the mock-treated HBECs (FIG. 3F, apoptotic cells indicated with arrows), which was not observed in those treated with ARINA-1 (FIG. 3H). In addition, mock-treated cells showed significant immunostaining using an antibody against the viral S glycoprotein (FIG. 3J), which again was not observed in the ARINA-1 treated cells (FIG. 3L). FIG. 3M provides comparison of the cilia length of the SARS-CoV2-exposed HBECs treated with saline or ARINA-1 demonstrated a significant shortening of cilia in the mock-treated cells but not in the ARINA 1-treated cells (ordinary one-way ANOVA, n=969 measured for each condition).

FIGS. 4A-4B show that ARINA-1 blocks SARS-CoV2 replication when administered after viral infection. FIG. 4A shows ARINA-1 applied 3 or 24 hours after exposing cells to virus significantly inhibited viral replication compared to mocked treated cells. Three independent experiments were done with at least 3 technical replicates per condition. FIG. 4B shows antiviral activity of the ARINA-1 components. Because ascorbic acid and glutathione alone are toxic to cells due to their acidities, ascorbic acid plus sodium bicarbonate and glutathione plus sodium bicarbonate at the same concentrations in ARINA-1 were assessed and demonstrated a significant inhibition of SARS-CoV2 replication compared to the vehicle. Bicarbonate alone did not show significant inhibition. Two independent experiments were performed with at least two technical replicates per condition. For both FIG. 4A and FIG. 4B RNA copy numbers were logarithmically transformed and compared using an ordinary one-way ANOVA statistical analysis.

FIGS. 5A-5C show ARINA-1 has no direct antiviral effect on SARS-CoV2 virus. FIG. 5A provides a flow diagram showing the procedure followed to test the direct antiviral of ARINA-1 on the virus. Briefly, a suspension of SARS-CoV2 virus was exposed to ARINA-1, incubated for 1 hour at 37° C., and then filtered through a 30,000 Da pore size membrane to remove the ARINA-1. The filter was washed three times with PBS 1× to remove any residual component of ARINA-1. The washed virus suspension was recovered and used to infect Vero E6 or HBE/ALI cells, and the viral load was determined after 48 h post infection. Virus particles were treated in parallel with saline as control. FIG. 5B shows ARINA-1-treated virus was as infectious as the mock-treated virus in Vero E6 cells. FIG. 5C shows ARINA-1-treated virus was as infectious as the mock-treated virus in the HBE/ALI assay. There were no significant differences between the ARINA-1-treated, mock-treated, and untreated viral particles using the Vero or HBECs. RNA copy numbers were logarithmically transformed and compared using ordinary one-way ANOVA statistical analysis.

FIGS. 6A-6B show ARINA-1 is not protective when cilia are not present in cells or when cilia beating is inhibited with BAPTA/AM. Undifferentiated 16HBE (FIG. 6A) cells are not protected by ARINA-1 when ciliary motility is absent or impaired. Two independent experiments with 3 replicates for Vero E6 and one experiment with 3 replicates for HBECs were performed. Data were logarithmically transformed and compared using Welch's t-test. FIG. 6B shows ARINA-1 antiviral activity is blocked by BAPTA-AM. The addition of ARINA-1 with BAPTA/AM does not rescue the antiviral activity of ARINA-1.

FIGS. 7A-7E show that the redox state of the cell is essential for the antiviral protection conferred by mucociliary transport. FIG. 7A shows that reduction of endogenous ROS production through the inhibition of xanthine oxidase with allopurinol (400 uM) significantly blocks SARS-CoV2 replication. FIG. 7B shows that the addition of the antioxidant agents NAC and sulforaphane also provided antiviral protection. FIG. 7C shows that none of the compounds showed cytotoxicity at the maximum concentrations tested in the previous experiment. Allopurinol (400 uM), NAC (50 mM) and sulphoraphane (SFN, 5 uM) showed negative values of toxicity indicating they preserved cell viability for the duration of the assay (72 hours). Viral loads are shown for ALI differentiated hNE cells from two WT donors (FIG. 7D) and from two PCD suffering human donors with mutations in genes CCDC39 and DNAI1 (FIG. 7E), which encode proteins essential for the assembly of dynein arm complexes and for dynein protein itself, respectively, treated with ARINA-1 or saline as control. Two experiments with at least two technical replicates for PCD-hNE cells and one experiment with four replicates for each WT-hNE cell donor were performed. Data were logarithmically transformed and compared using ordinary one-way ANOVA.

FIGS. 8A-8B show ARINA-1 induced a supernormal MCT and hyperciliation in HBECs. FIG. 8A and FIG. 8B show cilia beating frequency (CBF) and mucociliary transport (MCT) measurements using μOCT, respectively, in SARS-CoV-2-infected or uninfected, ARINA-1-treated or untreated HBEC. Comparisons were performed using an ordinary one-way ANOVA. MCT values were normalized against the MCT average of saline treated baseline controls.

FIGS. 8C-8F show resliced images of OCT captured videos in which the slope of the diagonal streak (arrow) indicates the vectorial transport of mucus particles over time. This allowed visualization of MCT rate on still images, in which higher slope angles with respect to time vectors are indicative of faster MCT rates.

FIG. 9 shows ARINA-1 inhibits the production of infectious virus by primary HBEC. Quantification of SARS-COV-2 virus using a luminescence TCID50 assay showed that ARINA-1-treated cells produced two order of magnitude less infectious virus that the saline-treated controls (TCID₅₀=1 vs 131-fold dilution, respectively). Values are averages and SD of three independent experiments, each using primary HBEC from a different donor. For each experiment, every dilution of the virus was assessed in sextuplicate using Vero E6 cells as indicators of cytotoxicity using a luminescence assay that measures ATP as a proxy for cell viability.

### DETAILED DESCRIPTION

### Definitions

To facilitate an understanding of the present invention, a number of terms and phrases are defined below.

As used in the present disclosure and claims, the singular forms “a,” “an,” and “the” include plural forms unless the context clearly dictates otherwise.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” as used herein means approximately ±10%. When the term “about” is used in conjunction with a numerical value or range, it modifies that value or range by extending the boundaries above and below the numerical values set forth. In general, the term “about” is used herein to modify a numerical value above and below the stated value by a variance of 10 percent, up or down (higher or lower), i.e., ±10%, unless a different variance is indicated (e.g., ±30%, ±20%, ±5%, ±1%, etc.).

“Clinical isolate” as used herein means a pathogen that has been isolated from a human subject or from a tissue sample taken from a human subject. “Clinical isolate bacteria” as used herein means a bacterial strain that has been isolated from a human subject or from a tissue sample taken from a human subject. “Clinical isolate virus” as used herein means a viral strain that has been isolated from a human subject or from a tissue sample taken from a human subject.

“Pharmaceutically acceptable” as used herein means safe and effective for use in humans. For example, a “pharmaceutically acceptable salt”, as used herein, means those salts of the compounds disclosed herein that are safe and effective for use in a subject and that possess the desired biological activity of the compound.

“Biofilm” as used herein means a group of microorganisms, e.g., clinical isolate bacteria, in which cells of the microorganism stick to each other and often these cells adhere to a surface. In some aspects, these adherent cells are embedded within a self-produced matrix of extracellular polymeric substance (EPS). In some aspects, the biofilm comprises a single bacterial species. In other aspects, the biofilm is a mixture of two or more species of bacteria.

“Extracellular” as used herein means outside a cell.

“Antibiotic resistance” refers to bacteria possessing a mechanism that makes an antibiotic ineffective at killing the bacteria (e.g., bacteria which are “antibiotic resistant”). Exemplary mechanisms include, e.g., destruction of the antibiotic, antibiotic-target modification, and restricted penetration and/or efflux of the antibiotic. In some aspects, the bacteria become antibiotic resistant due to a mutation.

“Synergistic effect” as used herein means an effect arising between two or more therapeutic agents, e.g., a composition disclosed herein and an antibiotic that produces an effect greater than the sum of the two or more therapeutic agent's individual effects.

“Inhibiting” as used herein means blocking or stopping, e.g., stopping bacterial growth.

“Reducing” as used herein means decreasing or lowering the amount of, e.g., lowering the amount of bacterial growth (e.g., as compared to a starting point or as compared between two or more groups).

“Treating” or “treatment” as used herein refers to partially or completely alleviating, ameliorating, improving, relieving, delaying onset of, inhibiting progression of, reducing severity of, and/or reducing incidence of one or more signs, symptoms or features of a disease.

By “subject” or “patient” is meant any subject, particularly a mammalian subject, for whom diagnosis, prognosis, or therapy is desired. In certain aspects, the mammal is a human subject. In other aspects, a subject is a human patient. In a particular aspect, a subject is a human patient in need of treatment.

Administration “in combination with” one or more further therapeutic agents includes simultaneous (concurrent) or consecutive administration in any order.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered serially, by alternation, or in parallel as separate formulations; or (3) by some other regimen. When delivered in alternation therapy, a synergistic effect can be attained when the compounds are administered or delivered sequentially, e.g., by different injections in separate syringes or by inhalation of one therapy and oral administration of a second therapy or vice versa.

The term “pharmaceutical formulation” refers to a preparation which is in such form as to permit the biological activity of the active ingredient or active ingredients to be effective, and which contains no additional components which are unacceptably toxic to a subject to which the formulation would be administered. The formulation can be sterile.

An “effective amount” of a composition or active agent as disclosed herein is an amount sufficient to carry out a specifically stated purpose. An “effective amount” can be determined empirically and in a routine manner, in relation to the stated purpose.

The term “therapeutically effective amount” refers to an amount of composition or active agent as disclosed herein effective to “treat” a disease or disorder in a subject.

The term “long COVID” as disclosed herein refers to new, recurring, or ongoing symptoms, inflammation and/or clinical findings at about four or more weeks after infection with SARS-CoV-2, the virus that causes COVID-19, sometimes after initial symptom recovery. Long COVID can occur in patients who have had varying degrees of illness during acute infection, including those who had mild or asymptomatic infections. Long COVID is also known as post COVID conditions, post-acute COVID-19, long-term effects of COVID, post-acute COVID syndrome, chronic COVID, long-haul COVID, late sequelae, and others.

In some aspects, the one or more symptoms or signs of infection comprise lung inflammation, fever, chills, cough, shortness of breath, difficulty breathing, fatigue, muscle aches, body aches, headache, reduction in ability to taste, reduction in ability to smell, sore throat, congestion, runny nose, nausea, vomiting, diarrhea, chest pain, confusion, inability to wake, inability to stay awake, discolored skin, discolored lips, or discolored nail beds.

### Compositions of the Disclosure

In some aspects, glutathione, a glutathione derivative, a glutathione conjugate, a pharmaceutically acceptable salt thereof, an organic acid (e.g., ascorbic acid), or any combination thereof can be combined with a pharmaceutical carrier or excipient and, optionally, other components to provide a composition of the present disclosure. In some aspects, the amount of glutathione, a glutathione derivative, a glutathione conjugate, pharmaceutically acceptable salt thereof, or any combination thereof, e.g., reduced glutathione, in a composition disclosed herein is about 30-90% by weight, about 30-85% by weight, about 30-80% by weight, about 30-75% by weight, about 30-70% by weight, about 30-65% by weight, about 30-60% by weight, about 30-55% by weight, about 30-50% by weight. In some aspects, the amount of glutathione, a glutathione derivative, a glutathione conjugate, pharmaceutically acceptable salt thereof, or any combination thereof, e.g., reduced glutathione, in a composition disclosed herein is 30-50% by weight.

In some aspects, a composition disclosed herein further comprises an organic acid. In some aspects, the organic acid is selected from the group of acids consisting of ascorbic, acetic, adipic, aspartic, benzenesulfonic, benzoic, butyric, camphorsulfonic, camsylic, carbonic, chlorobenzoic, cholic, citric, edetic, edisylic, estolic, ethanesulfonic, formic, fumaric, gluceptic, gluconic, glucuronic, glutamic, glycolic, glycolylarsanilic, hippuric, 1-hydroxy-2-naphthoic, isethionic, isobutyric, isonicotinic, lactic, lactobionic, maleic, malic, malonic, mandelic, methanesulfonic, mucic, muconic, napthalenesulfonic, nicotinic, oxalic, oleic, orotic, p-nitromethanesulfonic, pamoic, pantothenic, phthalic, polygalactouronic, propionic, saccharic, salicylic, stearic, suberic, succinic, sulfanilic, tannic, tartaric, p-toluenesulfonic and any combination thereof. In some aspects, the organic acid is ascorbic acid or a pharmaceutically acceptable salt thereof.

In some aspects, the amount of an organic acid, e.g., reduced ascorbic acid, in a composition disclosed herein is about 10-90% by weight, about 10-85% by weight, about 10-80% by weight, about 10-75% by weight, about 10-70% by weight, about 10-65% by weight, about 10-60% by weight, about 10-55% by weight, about 10-50% by weight, about 10-45% by weight, about 10-40% by weight, about 10-35% by weight, about 10-30% by weight, about 1-30% by weight, about 1-20% by weight, or about 1-10% by weight. In some aspects, the amount of an organic acid, e.g., reduced ascorbic acid, in a composition disclosed herein is 25-40% by weight.

In some aspects, a composition disclosed herein further comprises a bicarbonate salt. In some aspects, the bicarbonate salt is sodium bicarbonate. In some aspects, the amount of bicarbonate salt, e.g., sodium bicarbonate, in a composition disclosed herein is about 10-90% by weight, about 10-85% by weight, about 10-80% by weight, about 10-75% by weight, about 10-70% by weight, about 10-65% by weight, about 10-60% by weight, about 10-55% by weight, about 10-50% by weight, about 10-45% by weight, about 10-40% by weight, about 10-35% by weight, about 10-30% by weight, about 1-30% by weight, about 1-20% by weight, or about 1-10% by weight. In some aspects, the amount of bicarbonate salt, e.g., sodium bicarbonate, in a composition disclosed herein is about 20-30% by weight. In some aspects, a composition disclosed herein does not comprise a bicarbonate salt.

In some aspects, the pH of a composition disclosed herein is about 6.0 to about 8. In some aspects, the pH of a composition disclosed herein is greater than 5.5 or at least 6.0. (e.g., 5.6 to 14, 5.7 to 14, 5.8 to 14, 5.9 to 14, 6 to 14, 5.6 to 12, 5.7 to 12, 5.8 to 12, 5.9 to 12, 6 to 12, 5.6 to 10, 5.7 to 10, 5.8 to 10, 5.9 to 10, 6 to 10, 5.6 to 9, 5.7 to 9, 5.8 to 9, 5.9 to 9, 6 to 9, 5.6 to 8, 5.7 to 8, 5.8 to 8, 5.9 to 8, 6 to 8, 5.6 to 7.5, 5.7 to 7.5, 5.8 to 7.5, 5.9 to 7.5, 6 to 7.5, 5.6 to 7, 5.7 to 7, 5.8 to 7, 5.9 to 7, or 6 to 7).

In some aspects, a composition disclosed herein is formulated to maximize formulation stability and minimize oxidation of glutathione. Oxidized glutathione is associated with the generation of protein-carbonyls via glutathionlyation. Glutathionylation occurs when oxidized glutathione dissociates and attaches to proteins. Maintaining the glutathione in the reduced state in solution prior to administration can decrease the risk of glutathionylation products that can result in clinical complications such as bronchiectasis. In some aspects, the oxidized glutathione (e.g., % GSSG) in a composition disclosed herein is less than about 20%, less than about 18%, less than about 16%, less than about 15%, less than about 12%, less than about 10%, less than about 9%, less than about 8%, less than about 7%, less than about 6%, less than about 5%, less than about 4%, or less than about 3% by weight of the total glutathione in a composition disclosed herein after storage of a composition disclosed herein for 4 weeks (e.g., at 5° C. in a N₂ atmosphere and/or ambient atmosphere). In some aspects, the percentage of oxidized glutathione (e.g., % GSSG) in a composition disclosed herein is no more than about 2% to about 20%, about 2% to about 18%, about 2% to about 16%, about 2% to about 16%, about 2% to about 10%, or about 2% to 8% by weight of the total glutathione in a composition disclosed herein following 4 weeks of storage (e.g., at 5° C. in a N₂ atmosphere and/or ambient atmosphere). In some aspects, the percentage of oxidized glutathione (e.g., % GSSG) in a composition disclosed herein is less than about 20%, less than about 18%, less than about 16%, or less than about 10% by weight of the total glutathione in a composition disclosed herein following 4 weeks of storage (e.g., at 5° C. in a N₂ atmosphere and/or ambient atmosphere).

In some aspects, the reduced glutathione in a composition disclosed herein is more than about 80%, more than about 82%, more than about 84%, more than about 85%, more than about 88%, more than about 90%, more than about 91%, more than about 92%, more than about 93%, more than about 94%, more than about 95%, more than about 96%, or more than about 97% by weight of the total glutathione in a composition disclosed herein after storage of a composition disclosed herein for 4 weeks at about 5° C. (e.g., in a N₂ or ambient atmosphere). In some aspects, the percentage of reduced glutathione in a composition disclosed herein is between about 80% to about 100%, between about 80% to about 98%, between about 82% to about 98%, between about 84% to about 98%, between about 86% to about 98%, between about 88% to about 98%, between about 90% to about 98%, or between about 92% to to about 98% by weight of the total glutathione in a composition disclosed herein following 4 weeks of storage at 5° C. (e.g., in a N₂ or ambient atmosphere). In some aspects, the percentage of reduced glutathione in a composition disclosed herein is at least 80%, at least 82%, at least 84%, at least 86%, at least 88%, or at least 90% by weight of the total glutathione in a composition disclosed herein following 4 weeks of storage at 5° C. in a N₂ or ambient atmosphere.

In some aspects, a composition disclosed herein is further formulated to maximize formulation stability and minimize oxidation of an organic acid, e.g., ascorbic acid. Additionally, when ascorbic acid is oxidized into dehydroascorbate (DHA), DHA can break down and result in the formation of protein adducts in process called ascorbylation. Maintaining the organic acid, e.g., ascorbic acid, in the reduced state in solution prior to administration can decrease the risk of ascorbylation from the breakdown products of dehydroascorbate. In some aspects, the reduced ascorbic acid (e.g., % ASC) is more than about 80%, more than about 85%, more than about 86%, more than about 87%, more than about 88%, more than about 89%, or more than about 90% by weight of the ascorbic acid in a composition disclosed herein after storage of a composition disclosed herein for 4 weeks (e.g., at 5° C. in a N₂ atmosphere and/or ambient atmosphere). In some aspects, the percentage of reduced ascorbic acid (e.g., % ASC) in a composition disclosed herein is between about 82% to about 100% or between about 85% to about 95% by weight of the total ascorbic acid in a composition disclosed herein following 4 weeks of storage (e.g., at 5° C. in a N₂ atmosphere and/or ambient atmosphere). In some aspects, the percentage of reduced ascorbic acid (e.g., % ASC) in a composition disclosed herein is at least 80%, at least 85%, at least 86%, at least 87%, at least 88%, at least 89%, or at least 90% by weight of the total ascorbic acid in a composition disclosed herein following 4 weeks of storage (e.g., at 5° C. in a N₂ atmosphere and/or ambient atmosphere).

In some aspects, the oxidized ascorbic acid in a composition disclosed herein is less than about 20%, less than about 18%, less than about 16%, less than about 15%, less than about 12%, less than about 10%, or less than about 9% by weight of the total ascorbic acid in a composition disclosed herein after storage of a composition disclosed herein for 4 weeks (e.g., at 5° C. in a N₂ atmosphere and/or ambient atmosphere). In some aspects, the percentage of oxidized ascorbic acid in a composition disclosed herein is no more than about 5% to about 20%, about 5% to about 18%, about 5% to about 10%, or about 5% to 9% by weight of the total ascorbic acid in a composition disclosed herein following 4 weeks of storage (e.g., at 5° C. in a N₂ atmosphere and/or ambient atmosphere). In some aspects, the percentage of oxidized ascorbic acid in a composition disclosed herein is less than about 20%, less than about 18%, less than about 16%, or less than about 10% by weight of the total ascorbic acid in a composition disclosed herein following 4 weeks of storage (e.g., at 5° C. in a N₂ atmosphere and/or ambient atmosphere).

In certain aspects, the ratios of the components of a composition disclosed herein are formulated to maximize formulation stability and minimize oxidation of glutathione and an organic acid, e.g., ascorbic acid. In some aspects, glutathione and an organic acid (e.g., ascorbic acid) are formulated to comprise molar equivalents in solution, e.g., about 0.5-1:1, about 0.6-1:1, 0.7-1:1, 0.8-1:1, 0.9-1:1 or about 1:1 molar ratio of glutathione to ascorbic acid. In some aspects, the glutathione and an organic acid (e.g., ascorbic acid) are formulated to comprise molar excess of an organic acid (e.g., ascorbic acid) relative to glutathione in solution, e.g., about 1:1.1, about 1:1.2, about 1:3, about 1:4, about 1:5 molar ratio of glutathione to ascorbic acid.

In some aspects, a composition disclosed herein further comprises a bicarbonate salt (e.g., sodium bicarbonate). In some aspects, glutathione, an organic acid (e.g., ascorbic acid), and bicarbonate salt (e.g., sodium bicarbonate) are formulated to comprise a molar ratio of about 0.1-0.5:0.5-1:1, about 0.2-0.5:0.5-1:1, about 0.3-0.5:0.5-1:1, about 0.4-0.5:0.5-1:1, about 0.49:0.5-1:1, about 0.5:0.5-1:1, about 0.1-0.5:0.6-1:1, about 0.2-0.5:0.6-1:1, about 0.3-0.5:0.6-1:1, about 0.4-0.5:0.6-1:1, about 0.49:0.6-1:1, about 0.5:0.6-1:1, about 0.1-0.5:0.7-1:1, about 0.2-0.5:0.7-1:1, about 0.3-0.5:0.7-1:1, about 0.4-0.5:0.7-1:1, about 0.49:0.7-1:1, about 0.5: 0.7-1:1, about 0.1-0.5:0.8-1:1, about 0.2-0.5:0.8-1:1, about 0.3-0.5:0.8-1:1, about 0.4-0.5:0.8-1:1, about 0.49:0.8-1:1, about 0.5:0.8-1:1, about 0.1-0.5:0.9-1:1, about 0.2-0.5:0.9-1:1, about 0.3-0.5:0.9-1:1, about 0.4-0.5:0.9-1:1, about 0.49:0.9-1:1, about 0.5:0.9-1:1, about 0.1-0.5:1:1, about 0.2-0.5:1:1, about 0.3-0.5:1:1, about 0.4-0.5:1:1, about 0.49:1:1, about 0.5:1:1, molar ratio of glutathione to an organic acid (e.g., ascorbic acid) to bicarbonate salt (e.g., sodium bicarbonate). In some aspects, the molar ratio of glutathione, an organic acid (e.g., ascorbic acid), and bicarbonate salt (e.g., sodium bicarbonate) is 0.1-0.5:0.5-1:1, 0.4-0.5:0.5-1:1, 0.1-0.5:0.5:1, 0.1-0.5:1:1, or 0.4-0.5:1:1. In some aspects, the molar ratio of glutathione, an organic acid (e.g., ascorbic acid), and bicarbonate salt (e.g., sodium bicarbonate) is 0.49:0.5:1, 0.5:0.5:1, 0.49:1:1, or 0.5:1:1.

In some aspects, the bicarbonate salt (e.g., sodium bicarbonate) is less than the combined molar ratio of (a) glutathione, a glutathione derivative, a glutathione conjugate, a pharmaceutically acceptable salt thereof, or any combination thereof and (b) an organic acid (e.g., ascorbic acid). In some aspects, the molar ratio of glutathione, an organic acid (e.g., ascorbic acid), and bicarbonate salt (e.g., sodium bicarbonate) is 0.1-0.49:0.5:1, 0.2-0.49:0.5:1, 0.3-0.49:0.5:1, or 0.4-0.49:0.5:1.

In some aspects, a composition disclosed herein comprises or consists essentially of (a) a glutathione, a glutathione derivative, a glutathione conjugate, pharmaceutically acceptable salt thereof, or any combination thereof, and (b) an organic acid, wherein the molar ratio of (a) to (b) is about 0.5-1:1, about 0.6-1:1, 0.7-1:1, 0.8-1:1, 0.9-1:1 or about 1:1 and the pH of the composition is about 5.5 to 14, about 6 to about 8, 7±1.5, 6±0.5, or about 6.

In some aspects, a composition disclosed herein comprises or consists essentially of (a) a glutathione, a glutathione derivative, a glutathione conjugate, pharmaceutically acceptable salt thereof, or any combination thereof, (b) an organic acid, (c) a bicarbonate salt, wherein the molar ratio of (a) to (b) to (c) is about 0.1-0.5:0.5-1:1, 0.4-0.5:0.5-1:1, 0.1-0.5:0.5:1, 0.1-0.5:1:1, 0.4-0.5:1:1, 0.1-0.49:0.5:1, 0.2-0.49:0.5:1, 0.3-0.49:0.5:1, or 0.4-0.49:0.5:1 and the pH of the composition is about 5.5 to 14, about 6 to about 8, 7±1.5, 6±0.5, or about 6.

Pharmaceutical compositions for use in the present disclosure can be formulated using one or more physiologically acceptable carriers and/or excipients that facilitate administration of an organic acid, glutathione, a glutathione derivative, a glutathione conjugate, a pharmaceutically acceptable salt thereof, or any combination thereof to a subject by an intended route, e.g., delivery by inhalation. In some aspects, the pharmaceutical composition is an aqueous solution. In some aspects, the pharmaceutical composition is a dry powder.

A composition disclosed herein can be manufactured by conventional mixing, dissolving, granulating, dragee-making, emulsifying, encapsulating, entrapping, spray drying, or lyophilizing processes that are known in the art. The particular formulation depends upon the route of administration chosen. In one aspect, glutathione, a glutathione derivative, a glutathione conjugate, a pharmaceutically acceptable salt thereof, or any combination thereof is dissolved in a solvent, e.g., water, for administration to the airway of a subject (e.g., intranasal administration).

The term “pharmaceutically acceptable carrier” refers to a pharmaceutically acceptable material, composition, or vehicle, such as a liquid (e.g., water), or a solid filler, diluent, excipient, solvent, or encapsulating material. A carrier is “pharmaceutically acceptable” in the sense of being compatible with the other ingredients of a pharmaceutical formulation and suitable for use in humans without toxicity, irritation, allergic response, immunogenicity, or other complications commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

In some aspects, a composition disclosed herein comprise an excipient. In some aspects, the excipient is selected from the group consisting of a pH adjusting agent, a preservative, a chelating agent, and any combination thereof.

In certain aspects, a composition disclosed herein can comprise a pH adjusting agent. pH adjusting agents are known in the art. See, e.g., Remington's Pharmaceutical Sciences, 18th edition, A. R Gennaro, Ed., Mack Publishing Company (1990) and Handbook of Pharmaceutical Excipients, 3rd edition, A. Kibbe, Ed., Pharmaceutical Press (2000). Suitable examples of pharmaceutically acceptable pH adjusting agents include, but are not limited to, ascorbic acid, citric acid, sodium citrate, sodium bicarbonate, potassium bicarbonate, dibasic sodium phosphate, magnesium oxide, calcium carbonate, magnesium hydroxide, buffers (e.g., acetate buffers, citrate buffers, phosphate buffers, lactic acid buffers, and borate buffers, and any combination thereof), fat-soluble fatty acid esters of ascorbic acid (vitamin C) (e.g., alone or in combination with a-hydroxy acids), oxidation-resistant saturated fatty acid esters of ascorbic acid (e.g., ascorbyllaurate, ascorbyl myristate, ascorbyl palmitate, ascorbyl stearate, and ascorbyl behenate, and any combination thereof), and any combination thereof. In some aspects, esters can be prepared using hydrogenated oils or fats, or fractions thereof, and contain small amounts of another ester. Ascorbyl stearate prepared using canola, for example, can commonly contain about 4% ascorbyl palmitate.

In one aspect, the pH adjusting agent, e.g., ascorbic acid, is present in a composition disclosed herein in an amount of about 0.01-50% by weight, about 10-90% by weight, about 10-85% by weight, about 10-80% by weight, about 10-75% by weight, about 10-70% by weight, about 10-65% by weight, about 10-60% by weight, about 10-55% by weight, about 10-50% by weight, about 10-45% by weight, about 10-40% by weight, about 10-35% by weight, about 10-30% by weight, about 1-30% by weight, about 1-20% by weight, or about 1-10% by weight. In some aspects, the pH adjusting agent is present in a composition disclosed herein at an amount of about 1% by weight, about 5% by weight, about 10% by weight, about 15% by weight, about 20% by weight, about 25% by weight, about 30% by weight, about 35% by weight, about 40% by weight, about 45% by weight, or about 50% by weight of the composition.

In certain aspects, a composition disclosed herein can comprise preservatives. Pharmaceutically acceptable preservatives include, but are not limited to, various antibacterial and antifungal agents, solvents (e.g., ethanol, propylene glycol, benzyl alcohol and chlorobutanol, and any combination thereof), quaternary ammonium salts (e.g., cetylypridinium chloride, benzalkonium chloride and parabens including, but not limited to, methyl paraben, ethyl paraben and propyl paraben), chlorhexidine, benzoic acid and the salts thereof, parahydroxybenzoic acids and the salts thereof, alkyl esters of parahydroxybenzoic acid and the salts thereof, phenylmercuric salts such as nitrate, chloride, acetate, and borate, antioxidants, EDTA, sorbitol, phenol, boric acid and the salts thereof, sorbic acid and the salts thereof, thimerosal and nitromersol, and any combinations thereof.

In one aspect, the preservative is present in a composition disclosed herein in about 0.01-50% by weight, e.g., about 1-30% by weight, about 1-20% by weight, or about 1-10% by weight, e.g., about 1% by weight, about 5% by weight, about 10% by weight, about 15% by weight, about 20% by weight, about 25% by weight, about 30% by weight, about 35% by weight, about 40% by weight, about 45% by weight, or about 50% by weight of the composition.

In certain aspects, a composition disclosed herein can comprise a chelating agent. Non-limiting examples of chelating agents include lactic acid, acetic acid, oxalic acid, malonic acid, succinic acid, maleic acid, fumaric acid, aconitic acid, pimelic acid, sebacic acid, allymalonic acid, ethylmalonic acid, citric acid, malic acid, glyceric acid, tartaric acid, mevaloic acid, oxyglutaric acid, oxaloacetic acid, a-ketoglutaric acid, a-ketomalonic acid, glucuronic acid, galaceturonic acid, mannuronic acid, aspartic acid, glutamic acid, glycine, alanine, lysine, histidine, alginine, cysteine, s-aminocaproic acid, phenylalanine, phenylglycine, p-hydroxyphenylglycine, p-aminophenylalanine, y-carboxyglutamic acid, iminodiacetic acid, hydroxyethyliminodiacetic acid, ethylenediaminediacetic acid, ethylenediaminetetraacetic acid, trans-cyclohexane-diaminetetraacetic acid, diethylenediaminepentaacetic acid, alaninediacetic acid, diaminopimelic acid, phthalic acid, terephthalic acid, homophthalic acid, phenylsuccinic acid, phenylmalonic acid, oxanylic acid-o-carboxylic acid, anthralininoacetic acid, 2,4-dihydroxybenzoic acid, p-aminosalicyclic acid, phthalyglutamic acid, kynurenine, 1,2-hyroxybenzene-3,5-disulfonic acid, 4-amino-phenol-2-sulfonic acid, cysteic acid, 2-phosphoglyceric acid, glycero-3-phosphoric acid, glucose-1,6-diphosphoric acid, fructose-1,6-diphosphoric acid and phosphates (e.g., sodium phosphate, sodium aluminum phosphate, sodium acid phosphate, dipotassium phosphate, disodium phosphate, monobasic and sodium hexametaphosphate), and any combination thereof. Chelating agents can be included in the pharmaceutical compositions of this disclosure either as the parent molecule or in the salt form where appropriate. For example, compounds containing an acid function can be used in the protonated form or as a pharmaceutically acceptable inorganic or organic salt which retains the chelating activity of the parent compound

In one aspect, the chelating agent is present in a composition disclosed herein in about 0.01-50% by weight, e.g., about 1-30% by weight, about 1-20% by weight, or about 1-10% by weight, e.g., about 1% by weight, about 5% by weight, about 10% by weight, about 15% by weight, about 20% by weight, about 25% by weight, about 30% by weight, about 35% by weight, about 40% by weight, about 45% by weight, or about 50% by weight of the composition.

In certain aspects, a composition disclosed herein comprises glutathione, a glutathione derivative, a glutathione conjugate, a pharmaceutically acceptable salt thereof, or any combination thereof; a bicarbonate (e.g., sodium bicarbonate or potassium bicarbonate) and/or a pH modifier (e.g., ascorbic acid). In some aspects, a composition disclosed herein comprises glutathione, a glutathione derivative, a glutathione conjugate, a pharmaceutically acceptable salt thereof, or any combination thereof and/or a pH modifier such as an organic acid (e.g., ascorbic acid). In some aspects, the composition claimed wherein the amount of each component is present such that the amount of ascorbic acid is approximately molar equivalent or in molar excess of that of glutathione, a glutathione derivative, a glutathione conjugate, a pharmaceutically acceptable salt thereof, or any combination thereof.

In some aspects, a composition disclosed herein comprises (a) glutathione, a glutathione derivative, a glutathione conjugate, a pharmaceutically acceptable salt thereof; (b) an organic acid; and (c) a bicarbonate salt. In further aspects, the molar ratio of (a):(b):(c) in a composition disclosed herein is 0.1-0.5:0.5-1:1 (e.g., 0.4-0.5:0.5-1:1, 0.1-0.5:0.5:1, 0.1-0.5:1: 1, 0.4-0.5:1: 1, 0.1-0.49:0.5:1, 0.2-0.49:0.5:1, 0.3-0.49:0.5:1, or 0.4-0.49:0.5:1).

In some aspects, the organic acid in a Composition of the Disclosure is ascorbic acid. In some aspects, the bicarbonate salt in a composition disclosed herein is sodium bicarbonate. In some aspects, the composition comprises: (a) glutathione; (b) ascorbic acid; and (c) sodium bicarbonate. In certain aspects, the molar ratio of (a):(b):(c) is about 0.1-0.5:0.5-1:1 (e.g., about 0.49: about 0.50: about 1). In other aspects, the molar ratio of (a):(b):(c) is about 0.1-0.5:1: 1 (e.g., about 0.49: about 1: about 1).

In some aspects, the pH of a composition disclosed herein is from about 5.5 to about 14 (e.g. 5.5 to 7.5). In some aspects, the pH of a composition disclosed herein is from about 6 to about 14 (e.g., 6 to 7.5). In some aspects, the pH of the composition is 7±1.5, 7±1.4, 7±1.3, 7±1.2, 7±1.1, 6 0.5, 6 0.4, 6 0.3, 6 0.2, 6 0.5, 6 0.1, or about 6.

In some aspects, a composition disclosed herein is storage stable at 2-8° C. for at least 72 hours. In some aspects, a composition disclosed herein can (a) remain essentially free of precipitation after storage at 2-8° C. for at least 72 hours, (b) comprise less than 7%, less than 6%, less than 5%, or less than 4% impurities after storage at 2-8° C. for at least 72 hours, (c) have or maintain a pH from about 6 to 7.5 (e.g., 6.0-7.0) after storage at 2-8° C. for at least 72 hours, and/or (d) have minimal loss of solubility after storage at 2-8° C. for at least 72 hours.

In some aspects, the molar ratio of (a):(b) is about 0.1-0.5:0.5-1, about 0.1-0.6:0.5-1, about 0.1-0.7:0.5-1, about 0.1-0.8:0.5-1, about 0.1-0.9:0.5-1, about 0.1-1.0:0.5-1, about 0.1-1.1:0.5-1, about 0.1-1.2:0.5-1, about 0.1-1.3:0.5-1, about 0.1-1.4:0.5-1, about 0.1-1.5:0.5-1, about 0.1-1.6:0.5-1, about 0.1-1.7:0.5-1, about 0.1-1.8:0.5-1, about 0.1-1.9:0.5-1, about 0.1-2.0:0.5-1, about 0.1-0.5:0.5-1.1, about 0.1-0.5:0.5-1.2, about 0.1-0.5:0.5-1.3, about 0.1-0.5:0.5-1.4, about 0.1-0.5:0.5-1.5, about 0.1-0.5:0.5-1.6, about 0.1-0.5:0.5-1.7, about 0.1-0.5:0.5-1.8, about 0.1-0.5:0.5-1.9, or about 0.1-0.5:0.5-2.0. In some aspects, the molar ratio of (a):(b) is about 0.1-0.5:0.5-1.

In some aspects, the composition further comprises (c) bicarbonate or a pharmaceutically acceptable salt thereof. In some aspects, the composition comprises sodium bicarbonate or calcium bicarbonate.

In some aspects, the molar ratio of (a):(b):(c) is about 0.1-0.6:0.5-1:1.

In some aspects, the molar ratio of (a):(c) is about 0.1-0.5:0.5-1, about 0.1-0.6:0.5-1, about 0.1-0.7:0.5-1, about 0.1-0.8:0.5-1, about 0.1-0.9:0.5-1, about 0.1-1.0:0.5-1, about 0.1-1.1:0.5-1, about 0.1-1.2:0.5-1, about 0.1-1.3:0.5-1, about 0.1-1.4:0.5-1, about 0.1-1.5:0.5-1, about 0.1-1.6:0.5-1, about 0.1-1.7:0.5-1, about 0.1-1.8:0.5-1, about 0.1-1.9:0.5-1, about 0.1-2.0:0.5-1, about 0.1-0.5:0.5-1.1, about 0.1-0.5:0.5-1.2, about 0.1-0.5:0.5-1.3, about 0.1-0.5:0.5-1.4, about 0.1-0.5:0.5-1.5, about 0.1-0.5:0.5-1.6, about 0.1-0.5:0.5-1.7, about 0.1-0.5:0.5-1.8, about 0.1-0.5:0.5-1.9, or about 0.1-0.5:0.5-2.0.

In some aspects, the present disclosure provides a composition suitable for treatment or prevention of an infection with a pathogen. In some aspects, the pathogen is a coronavirus. In some aspects, the pathogen is SARS-CoV-2. In some aspects, SARS-CoV-2 is a variant is selected from the group consisting of an Alpha variant (e.g., B.1.1.7 and Q lineages), a Beta variant (e.g., B.1.351 and descendent lineages), a Gamma variant (e.g., P.1 and descendent lineages), a Delta variant (e.g., B.1.617.2 and AY lineages), an Epsilon variant (e.g., B.1.427 and B.1.429), an Eta variant (e.g., B.1.525), an Iota variant (e.g., B.1.526), a Kappa variant (e.g., B.1.617.1), 1.617.3, a Mu variant (e.g., B.1.621, B.1.621.1), a Zeta variant (e.g., P.2), or an Omicron variant (e.g., B.1.1.529, BA.1, BA.1.1, BA.2, BA.3, BA.4 and BA.5 lineages).

In some aspects, the composition comprises glutathione or a pharmaceutically acceptable salt thereof. In some aspects, the composition comprises a glutathione derivative or a pharmaceutically acceptable salt thereof. In some aspects, the composition comprises a glutathione conjugate or a pharmaceutically acceptable salt thereof.

In some aspects, the organic acid is ascorbic acid or a pharmaceutically acceptable salt thereof.

In some aspects, the composition is administered to the lungs by an inhalable dosage form. In some aspects, the inhalable dosage form is a metered dose inhaler, a dry powder inhaler, or a nebulizer.

In some aspects, the subject has long COVID.

In some aspects, the subject has developed one or more SARS-CoV-2-related sequelae. In some aspects, the one or more SARS-CoV-2-related sequelae comprises a bronchiectasis. In some aspects, the bronchiectasis is traction bronchiectasis. In some aspects, the bronchiectasis is non-cystic fibrosis bronchiectasis. In some aspects, the one or more SARS-CoV-2-related sequelae is characterized by a symptom selected from one or more of ageusia, myalgia, arthralgia, parosmia, anosmia, fatigue, headache, cough, chills, shivers, fever, dyspnea, sore throat, rhinorrhea, diarrhea, brain fog, nausea, subjective fever, abdominal pain, vomiting, rash, skin abnormality, and blood clots.

In some aspects, the subject has a chronic airway disease or condition. In some aspects, the chronic airway disease or condition is a pre-existing condition.

In some aspects, the subject belongs to a subject population having an increased risk of infection by a pathogen or an increased risk of a severe infection by a pathogen. In some aspects, the pathogen is SARS-CoV-2.

In some aspects, the subject is aged 50, 55, 60, 65, 70, 75, 80, 85, 90, 95, 100, or greater.

In some aspects, the subject suffers from an acute infection by a pathogen. In some aspects, the pathogen is SARS-CoV-2.

As used herein, glutathione or “GSH” can refer to a compound having the Formula A:

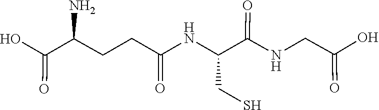 [SMILES: N[C@@H](CCC(=O)N[C@@H](CS)C(=O)NCC(=O)O)C(=O)O]

or a zwitterionic form thereof, e.g., a compound having the Formula B:

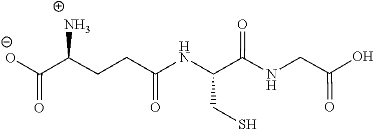 [SMILES: [NH3+][C@@H](CCC(=O)N[C@@H](CS)C(=O)NCC(=O)O)C(=O)[O-]]

In some aspects referred to as “Embodiment I,” the present disclosure provides a preparation comprising an aqueous solution in a closed container with a headspace, wherein:
(i) the aqueous solution comprises a salt having Formula I:

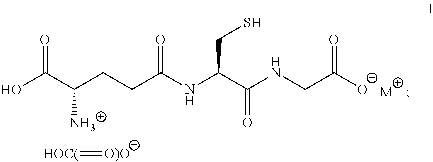

(ii) the atmosphere of the headspace comprises 90% or more carbon dioxide by volume; and
(iii) M⁺ is Na⁺, Li⁺, K⁺ or Cs⁺.

In another aspect, the aqueous solution of Embodiment I has a pH of 6.0±0.4 for 24 hours or more at about 5° C.

In another aspect, the aqueous solution of Embodiment I has a pH of 6.0±0.3 for 24 hours or more at about 5° C.

In another aspect, the aqueous solution of Embodiment I has a pH of 6.0±0.2 for 24 hours or more at about 5° C.

In another aspect, the aqueous solution of Embodiment I has a pH of 6.0±0.1 for 24 hours or more at about 5° C.

In another aspect, the aqueous solution of Embodiment I further comprises a salt having Formula II:

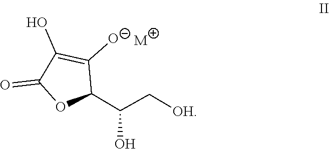

In another aspect, the aqueous solution of Embodiment I further comprises a salt having Formula III:

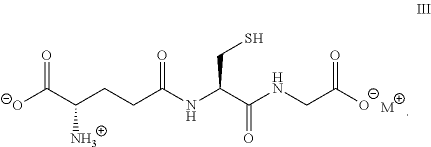

In another aspect, the atmosphere of the headspace of Embodiment I comprises 80% or more of carbon dioxide by volume.

In another aspect, the atmosphere of the headspace of Embodiment I comprises 85% or more of carbon dioxide by volume.

In another aspect, the atmosphere of the headspace of Embodiment I comprises 90% or more of carbon dioxide by volume.

In another aspect, the atmosphere of the headspace of Embodiment I comprises 95% or more of carbon dioxide by volume.

In another aspect, the aqueous solution of Embodiment I comprises about 10 wt % to about 20 wt % of the salt having Formula I.

In another aspect, the aqueous solution of Embodiment I comprises about 13 wt % to about 17 wt % of the salt having Formula I.

In another aspect, the aqueous solution of Embodiment I comprises about 14.7 wt % of the salt having Formula I.

In another aspect, the aqueous solution of Embodiment I comprises about 5 wt % to about 15 wt % of the salt having Formula II.

In another aspect, the aqueous solution of Embodiment I comprises about 7 wt % to about 11 wt % of the salt having Formula II.

In another aspect, the aqueous solution of Embodiment I comprises about 9.1 wt % of the salt having Formula II.

In another aspect, the aqueous solution of Embodiment I has a density of about 1.13 g/L.

In another aspect, the aqueous solution of Embodiment I is frozen.

In another aspect, M⁺ is Na⁺ in Embodiment I.

In another aspect, M⁺ is Li⁺ in Embodiment I.

In another aspect, M⁺ is K⁺ in Embodiment I.

In another aspect, M⁺ is Cs⁺ in Embodiment I.

In another embodiment, the preparation of Embodiment I is packaged as a single unit dose. In another aspect, the single unit dose is in a sealed vial.

In another aspect, the preparation of Embodiment I is marketed, distributed, or administered as part of a pharmaceutical product.

In some aspects, the preparation of Embodiment I can further comprise any one or more of the further aspects disclosed herein.

In another aspect referred to as “Embodiment II,” the present disclosure provides a method of making the preparation of Embodiment I (or Embodiment I including one or more the further aspects disclosed above), the method comprising:
(i) dissolving L-glutathione, ascorbic acid, and M⁺HCO₃⁻, wherein M⁺ is Na⁺, Li⁺, K⁺ or Cs⁺, in water for injection under carbon dioxide to give an aqueous solution;
(ii) transferring a portion of the aqueous solution to a container;
(iii) overlaying the aqueous solution with carbon dioxide; and
(iv) sealing the container with a stopper.

In another embodiment, about 8 wt % to about 18 wt % of L-glutathione, about 3 wt % to about 13 wt % of ascorbic acid, and about 3 wt % to about 13 wt % of M⁺HCO₃⁻ is dissolved in about 62 wt % to about 82 wt % water for injection to give the aqueous solution of Embodiment II.

In another aspect, the aqueous solution of Embodiment II has a pH of 6.0±0.4 for 24 hours or more at about 5° C.

In another aspect, the aqueous solution of Embodiment II has a pH of 6.0±0.3 for 24 hours or more at about 5° C.

In another aspect, the aqueous solution of Embodiment II has a pH of 6.0±0.2 for 24 hours or more at about 5° C.

In another aspect, the aqueous solution of Embodiment II has a pH of 6.0±0.1 for 24 hours or more at about 5° C.

In another aspect, M⁺ is Na⁺, i.e., M⁺ HCO₃⁻ is sodium bicarbonate, in Embodiment II.

In another aspect, M⁺ is Li⁺, i.e., M⁺ HCO₃⁻ is lithium bicarbonate, in Embodiment II.

In another aspect, M⁺ is K⁺, i.e., M⁺ HCO₃⁻ is potassium bicarbonate, in Embodiment II.

In another aspect, M⁺ is Cs⁺, i.e., M⁺ HCO₃⁻ is cesium bicarbonate, in Embodiment II.

In another aspect, about 11 wt % to about 15 wt % of L-glutathione, about 5 wt % to about 9 wt % of ascorbic acid, and about 5 wt % to about 9 wt % of sodium bicarbonate is dissolved in about 68 wt % to about 76 wt % water to give the aqueous solution of Embodiment II, wherein M⁺ is Na⁺.

In another aspect, about 13.0 wt % of L-glutathione, about 7.6 wt % of ascorbic acid, and about 7.3 wt % of sodium bicarbonate is dissolved in about 72.0 wt % water to give the aqueous solution of Embodiment II, wherein M⁺ is Na⁺.

In some aspects, the method of Embodiment II can further comprise any one or more of the further aspects disclosed herein.

In another aspect referred to as “Embodiment III,” the present disclosure provides an aqueous solution comprising a salt having Formula I.

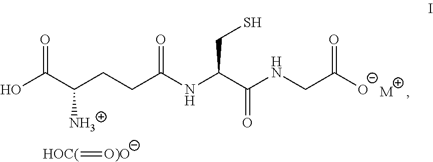

prepared by dissolving L-glutathione, ascorbic acid, and M⁺HCO₃⁻, wherein M⁺ is Na⁺, Li⁺, K⁺ or Cs⁺, in water for injection under an atmosphere of carbon dioxide.

In another aspect, about 8 wt % to about 18 wt % L-glutathione, about 3 wt % to about 13 wt % of ascorbic acid, and about 3 wt % to about 13 wt % M⁺HCO₃⁻ is dissolved in about 62 wt % to about 82 wt % water for injection to give the aqueous solution of Embodiment III.

In another aspect, the aqueous solution of Embodiment III has a pH of 6.0±0.4 for 24 hours or more at about 5° C.

In another aspect, the aqueous solution of Embodiment III has a pH of 6.0±0.3 for 24 hours or more at about 5° C.

In another aspect, the aqueous solution of Embodiment III has a pH of 6.0±0.2 for 24 hours or more at about 5° C.

In another aspect, the aqueous solution of Embodiment III has a pH of 6.0±0.1 for 24 hours or more at about 5° C.

In another aspect, the aqueous solution of Embodiment III further comprises a salt having Formula II:

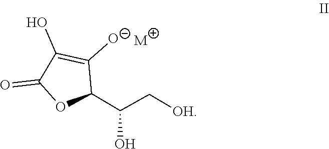

In another aspect, the aqueous solution of Embodiment III further comprises a salt having Formula III:

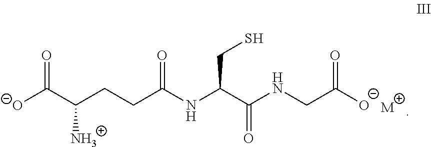

In another aspect, M⁺ is Na⁺, i.e., M⁺ HCO₃⁻ is sodium bicarbonate, in Embodiment III.

In another aspect, M⁺ is Li⁺, i.e., M⁺ HCO₃⁻ is lithium bicarbonate, in Embodiment III.

In another aspect, M⁺ is K⁺, i.e., M⁺ HCO₃⁻ is potassium bicarbonate, in Embodiment III.

In another aspect, M⁺ is Cs⁺, i.e., M⁺ HCO₃⁻ is cesium bicarbonate, in Embodiment III.

In another aspect, about 11 wt % to about 15 wt % L-glutathione, about 5 wt % to about 9 wt % of ascorbic acid, and about 5 wt % to about 9 wt % sodium bicarbonate is dissolved in about 68 wt % to about 76 wt % water to give the aqueous solution in Embodiment III.

In another aspect, about 13.0 wt % L-glutathione, about 7.6 wt % ascorbic acid, and about 7.3 wt % sodium bicarbonate is dissolved in about 72.0 wt % water to give the aqueous solution in Embodiment III.

In some aspects, the aqueous solution of Embodiment III can further comprise any one or more of the further aspects disclosed herein.

Glutathione plays a role in the detoxification of xenobiotic compounds and in the antioxidation of reactive oxygen species and free radicals. See, e.g., Bray and Taylor, Canadian Journal of Physiology and Pharmacology 71:746-751 (1993).

In individuals with chronic inflammatory airway diseases, such as lung transplant patients or patients having received other transplants (e.g., solid organ, blood, bone marrow), glutathione reserves are depleted.

In some aspects, the composition comprises a glutathione-containing conjugate or a pharmaceutically acceptable salt thereof. In certain aspects, the glutathione-containing conjugate is metabolized to release glutathione, or a derivative thereof, upon administration to a subject.

In one aspect, a glutathione conjugate is a compound having Formula I:

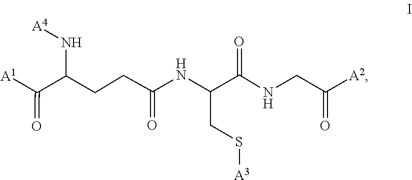

and the pharmaceutically acceptable salts and solvates thereof, wherein,
A¹ is —OR¹; A² is Z¹; A³ is hydrogen; and A⁴ is R^{3a}; or
A¹ is Z¹; A² is —OR²; and A³ is hydrogen; and A⁴ is R^{3a}; or
A¹ is —OR¹; A² is —OR²; and A³ is Z³; and A⁴ is R^{3a}; or
A¹ is Z²; A² is —OR²; and A³ is hydrogen; and A⁴ is R^{3a}; or
A¹ is —OR¹; A² is Z²; and A³ is hydrogen; and A⁴ is R^{3a}; or
A¹ is —OR¹; A² is —OR²; A³ is hydrogen; and A⁴ is Z³; or
A¹ and A² are each Z¹, and A³ is hydrogen;
Z¹ is selected from the group consisting of:

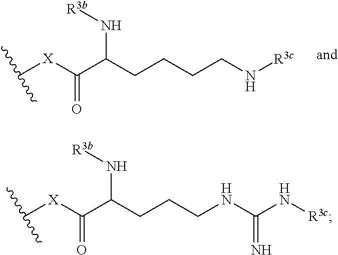

Z² is selected from the group consisting of:

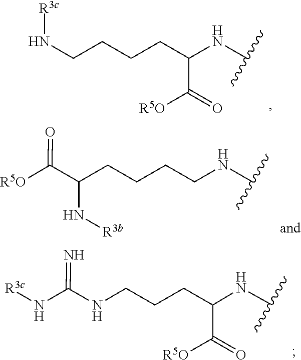

Z³ is selected from the group consisting of:

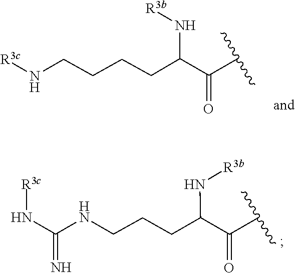

R¹ is selected from the group consisting of hydrogen and optionally substituted alkyl;
R² is selected from the group consisting of hydrogen and optionally substituted alkyl;
R^{3a}, R^{3b}, and R^{3c} are each independently selected from the group consisting of hydrogen and protecting group;
X is selected from the group consisting of:
—O—;
—O(CH₂)ₘO—;
—OCH₂CH(R⁴)O—;
—OCH(R⁴)CH₂O—; and
—O(CH₂CH₂O)ₙ—;
R⁴ is:

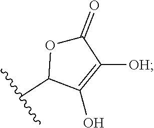 [SMILES: *C1OC(=O)C(O)=C1O]

m is 1, 2, 3, 4, 5, 6, 7, or 8;
n is 2, 3, 4, 5, 6, 7, or 8; and
R⁵ is selected from the group consisting of hydrogen and optionally substituted alkyl.

In another aspect, a glutathione conjugate is a compound having Formula I, and the pharmaceutically acceptable salts and solvates thereof, wherein m is 2, 3, 4, 5, 6, 7, or 8.

In another aspect, a glutathione conjugate is a compound having Formula II:

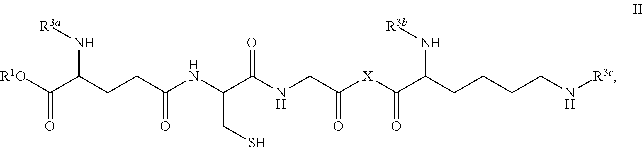

or a pharmaceutically acceptable salt or solvate thereof, wherein R1, R3a, R3b, R3c, and X are as defined in connection with Formula I.

In another aspect, a glutathione conjugate is enantiomerically enriched.

### Methods of Use

Certain aspects of the disclosure are directed to use of a composition disclosed herein is useful for treating, reducing the symptoms of, or preventing a disease, condition, or disorder of the lung associated with a viral infection, e.g., a SARS-CoV-2 viral infection. Certain aspects of the disclosure are directed to use of a composition disclosed herein is useful for upregulating mucociliary clearance in a subject suffering from or at risk of impaired mucociliary clearance, e.g., in a subject with or without an active SARS-CoV-2 infection in the lung. Certain aspects of the disclosure are directed to a method of protecting the lungs of a subject suffering from SARS-CoV-2 from cilia damage, e.g., shortening of cilia in the lung of the subject.

In some aspects, the subject suffers from a SARS-CoV-2 infection or has recently (e.g., within 2 weeks to 6 months) suffered from a SARS-CoV-2 infection. In some aspects, the subject is suffering from long COVID. In some aspects, the subject is suffering from inflammation incident to a SARS-CoV-2 infection. In some aspects, the subject is dependent on supplemental oxygen. In some aspects, the subject has reduced cilial function, expression, and/or height. In some aspects, the subject has lung inflammation, e.g., associated with current or previous COVID.

In some aspects, the subject suffers from long COVID. In some aspects, the subject suffers from one or more COVID symptoms at least four or more weeks after infection with SARS-CoV-2. In some aspects, the subject suffers from COVID symptoms after initial symptom recovery.

In some aspects, the one or more long COVID symptoms comprise one or more of lung inflammation, fever, chills, cough, shortness of breath, difficulty breathing, fatigue, muscle aches, body aches, headache, reduction in ability to taste, reduction in ability to smell, sore throat, congestion, runny nose, nausea, vomiting, diarrhea, chest pain, confusion, inability to wake, inability to stay awake, discolored skin, discolored lips, bronchiectasis or traction bronchiectasis, or discolored nail beds.

In some aspects, administering the composition upregulates mucus clearance without resulting in neutrophilia.

In some aspects, administration of the composition according to the methods disclosed herein blocks SARS-CoV-2 replication. In some aspects, administration of the composition according to the methods disclosed herein reduces SARS-CoV-2 viral load. In some aspects, administration of the composition according to the methods disclosed herein does not decrease SARS-CoV-2 infectivity.

In some aspects, the subject has a pre-existing pulmonary or airway disease or disorder, e.g., prior to suffering from one or more COVID symptoms or infection with SARS-CoV-2. In some aspects, the pulmonary or airway disorder is selected from the group consisting of chronic inflammatory lung disease, pulmonary fibrosis, pulmonary vasculitis, pulmonary sarcoidosis, inflammation and/or infection associated with lung transplantation, acute or chronic lung rejection and/or dysfunction, solid organ transplant, blood transplant, bone marrow transplant, restrictive airways disease, airway restriction, pulmonary artery hypertension, bronchitis, sinusitis, asthma, cystic fibrosis, bronchiectasis (e.g., non-cystic fibrosis bronchiectasis or cystic fibrosis bronchiectasis), bacterial infection, fungal infection, parasite infection, viral infection, chronic obstructive pulmonary disease (COPD), bronchiolitis obliterans syndrome (BOS), primary ciliary dyskinesia (PCD), alveolar protienosis, idiopathic or other pulmonary fibrosis, eosinophilic pneumonia, eosinophilic bronchitis, acute respiratory distress syndrome (ARDS), inflammation and/or infection associated with mechanical ventilation, ventilator-associated pneumonia, complications associated with an acute or chronic tracheostomy, traction bronchiectasis, chronic or recurrent pulmonary infection, asbestos-related airway disorder or disease, dust-related airway disorder or disease, silicosis, and radiation or chemical agent-related airway disease or disorder, and any combination thereof.

In some aspects, the pulmonary or airway disease or disorder is selected from the group consisting of chronic inflammatory lung disease, an inflammation and/or infection associated with lung transplantation, acute or chronic lung rejection or dysfunction, asthma, cystic fibrosis, bronchiectasis (e.g., non-cystic fibrosis bronchiectasis or cystic fibrosis bronchiectasis), or chronic obstructive pulmonary disease (COPD), or any combination thereof. In another aspect, the pulmonary or airway disease or disorder is cystic fibrosis. In another aspect, the subject is a lung transplant patient. In another aspect, the subject is a patient with non-cystic fibrosis bronchiectasis. In another aspect, the subject is infected with a biofilm producing bacteria.

In some aspects, the pulmonary or airway disease or disorder is bronchiectasis (BrE). Bronchiectasis can be diagnosed in patients suffering from cystic fibrosis (CF bronchiectasis) or patients without cystic fibrosis (non-CF bronchiectasis). To date, therapies that have been used in cystic fibrosis (CF) have not worked and have been shown to be potentially harmful in non-CF bronchiectasis.

Non-CF bronchiectasis and CF disease, including CF-associated bronchiectasis, are clinically distinct although they do have some of the same features. CF BrE affects primarily the upper lobes of the airways, whereas non-CF BrE is associated with more lower lobe involvement. Because non-CF BrE patients lack adequate mucociliary transport mechanisms, therapies that only break up components of mucus (e.g., DNase) cause sputum to pool in the lower airways and the lung parachyma rather than being expectorated. Sputum in CF contains more DNA compared to BrE patients, indicating distinct underlying physiologies. BrE patients are generally older with a higher prevalence in women, and CF patients are diagnosed at an early age and include equal male/female prevalence. (O'Donnell et al., Chest 1998 Volume 113, Issue 5, Pages 1329-1334).

In another aspect, the present disclosure provides methods of treating or preventing one or more COVID symptoms or infection with SARS-CoV-2 in the airway of a subject in need thereof, the method comprising administering to the subject a therapeutically effective amount of a composition disclosed herein. In another aspect, a composition disclosed herein is administered to the subject in combination with one or more anti-viral agents. The anti-viral agents can be administered locally to the lungs and/or systemically.

In another aspect, the present disclosure provides methods of treating or preventing inflammation in the airway of a subject in need thereof, the method comprising administering to the subject a therapeutically amount of a composition disclosed herein.

In another aspect, the present disclosure provides methods of treating or preventing a disease or disorder in mucosal tissue of a subject suffering from one or more COVID symptoms or infection with SARS-CoV-2, the method comprising administering to the subject a therapeutically effective amount of a composition disclosed herein. Non-limiting examples of mucosal tissue include the mouth, nose, eye, ear, upper respiratory tract, lower respiratory tract, gastrointestinal tract, vagina, rectum and urethra.

In another aspect, the present disclosure provides methods of treating or preventing a disease or disorder associated with mucosal membranes, in a subject suffering from one or more COVID symptoms or infection with SARS-CoV-2, the method comprising administering to the subject a therapeutically amount of a composition disclosed herein to the appropriate mucosal membranes. In one aspect, the mucosal membranes are the lungs, such as the deep lung (alveolar region) or in the lung parenchyma.

In another aspect, a composition disclosed herein can be used to treat a subject suffering from one or more COVID symptoms or infection with SARS-CoV-2, wherein the subject has a pre-existing conditions such as bronchiolitis obliterans and military-related lung damage, i.e., lung damage of military personnel who have damaged airways secondary to unknown exposures.

In another aspect, the present disclosure provides the use of a composition disclosed herein for the manufacture of a medicament for treatment of a pulmonary or airway disorder associated with COVID (e.g., long COVID) or an infection with SARS-CoV-2. In another aspect, the use further comprises administering one or more additional therapeutic agents to the subject (an anti-viral agent).

The therapeutic methods of this disclosure comprise administering a therapeutically effective amount of a composition disclosed herein to a subject in need thereof, e.g., a human patient. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

In another aspect, the subject suffering from one or more COVID symptoms or infection with SARS-CoV-2 has a pre-existing pulmonary or airway disorder or disease selected from the group consisting of chronic inflammatory lung disease, an inflammation and/or infection associated with lung transplantation, acute lung rejection, asthma, cystic fibrosis, bronchiectasis (e.g., non-cystic fibrosis bronchiectasis or cystic fibrosis bronchiectasis), tracheostomy, parenchymal inflammation, traction bronchiectasis, restrictive airways diseases, and chronic obstructive pulmonary disease (COPD), and any combination thereof.

In another aspect, the subject suffering from one or more COVID symptoms or infection with SARS-CoV-2 is treated by restoring homeostasis to and/or maintaining homeostasis in a mucosal membrane of a subject in need thereof, the method comprising administering to the subject an effective amount of a composition disclosed herein.

In another aspect, the present disclosure provides a method of restoring homeostasis to and/or maintaining homeostasis in a mucosal membrane of a subject suffering from one or more COVID symptoms or infection with SARS-CoV-2, the method comprising administering to the subject an effective amount of a composition disclosed herein.

In another aspect, the present disclosure provides a method of restoring or maintaining homeostasis in a mucosal membrane comprising administering to the subject suffering from one or more COVID symptoms or infection with SARS-CoV-2 an effective amount of a composition disclosed herein.

In some aspects, the methods of the disclosure comprise administering to the airway of the subject an effective amount of a composition comprising: a glutathione conjugate and/or glutathione, or a pharmaceutically acceptable salt thereof. In another aspect, the composition further comprises: an organic acid, or a pharmaceutically acceptable salt thereof. In another aspect, the organic acid is ascorbic acid. In another aspect, the composition further comprises: a bicarbonate salt. In another aspect, the bicarbonate salt is sodium bicarbonate or potassium bicarbonate. In some aspects, the organic acid in a Composition of the Disclosure is ascorbic acid. In some aspects, the bicarbonate salt in a composition disclosed herein is sodium bicarbonate. In some aspects, the composition comprises: (a) glutathione; (b) ascorbic acid; and (c) sodium bicarbonate. In certain aspects, the molar ratio of (a):(b):(c) is about 0.1-0.5:0.5-1:1 (e.g., about 0.49: about 0.50: about 1). In other aspects, the molar ratio of (a):(b):(c) is about 0.1-0.5:1: 1 (e.g., about 0.49: about 1: about 1).

In another aspect, the amount of each of component the glutathione conjugate and/or the glutathione, or a pharmaceutically acceptable salt thereof, the organic acid, or a pharmaceutically acceptable salt thereof, and the bicarbonate salt of the composition is present such that the amount of bicarbonate salt results in a pH in a range from about 5.5 to about 14. In some aspects, the pH of a composition disclosed herein is from about 5.5 to about 14 (e.g. 5.5 to 7.5). In some aspects, the pH of a composition disclosed herein is from about 6 to about 14 (e.g., 6 to 7.5). In some aspects, the pH of the composition is 7±1.5, 7±1.4, 7±1.3, 7±1.2, 7±1.1, 6±0.5, 6±0.4, 6±0.3, 6±0.2, 6±0.5, 6±0.1, or about 6.

In another aspect, the composition further comprises from about 0.01% to about 5% by weight of a pharmaceutically acceptable thiocyanate salt.

In another aspect, the composition is in the form of a particle. In another aspect, the particle is mixed with a gas or liquid propellant for use in an inhalation therapy. In some aspects, the inhalation therapy comprises administration of a nebulized formulation. In some aspects, the inhalation therapy comprises administration of a dry powder formulation.

In another aspect, the glutathione is reduced glutathione.

In another aspect, administration of the composition to the subject gives a concentration of about 0.1 mM to about 1.0 mM glutathione in the airway surface liquid of the subject. In another aspect, administration of the composition to the subject gives a concentration of about 0.5 mM to about 3.0 mM thiocyanate in the airway surface liquid of the subject.

In some aspects, the oxidized glutathione (e.g., % GSSG) in a composition disclosed herein is less than about 20%, less than about 18%, less than about 16%, less than about 15%, less than about 12%, less than about 10%, less than about 9%, less than about 8%, less than about 7%, less than about 6%, less than about 5%, less than about 4%, or less than about 3% by weight of the total glutathione in a composition disclosed herein after storage of a composition disclosed herein for 4 weeks (e.g., at 5° C. in a N₂ atmosphere and/or ambient atmosphere).

In some aspects, the reduced glutathione in a composition disclosed herein is more than about 80%, more than about 82%, more than about 84%, more than about 85%, more than about 88%, more than about 90%, more than about 91%, more than about 92%, more than about 93%, more than about 94%, more than about 95%, more than about 96%, or more than about 97% by weight of the total glutathione in a composition disclosed herein after storage of a composition disclosed herein for 4 weeks at about 5° C. (e.g., in a N₂ or ambient atmosphere).

In some aspects, the reduced ascorbic acid (e.g., % ASC) is more than about 80%, more than about 85%, more than about 86%, more than about 87%, more than about 88%, more than about 89%, or more than about 90% by weight of the ascorbic acid in a composition disclosed herein after storage of a composition disclosed herein for 4 weeks (e.g., at 5° C. in a N₂ atmosphere and/or ambient atmosphere).

In some aspects, the oxidized ascorbic acid in a composition disclosed herein is less than about 20%, less than about 18%, less than about 16%, less than about 15%, less than about 12%, less than about 10%, or less than about 9% by weight of the total ascorbic acid in a composition disclosed herein after storage of a composition disclosed herein for 4 weeks (e.g., at 5° C. in a N₂ atmosphere and/or ambient atmosphere).

In some aspects, the composition is an aqueous solution or a dry powder. In another aspect, the composition is administered by inhalation to the subject.

Certain aspects of the disclosure are directed to methods of increasing mucosilary clearance in the lung of a subject suffering from a SARS-CoV-2 infection. Mucociliary clearance is critical in preventing infections and inflammation in the lung. However, mucociliary clearance is often absent or impaired in chronic inflammatory airways diseases such as cystic fibrosis, bronchiectasis (e.g., non-cystic fibrosis bronchiectasis or cystic fibrosis bronchiectasis), lung transplant and chronic obstructive pulmonary disease. Moreover, airway epithelial cells from cystic fibrosis patients are known to completely lack mucociliary clearance ability without other inflammatory insults or bacterial colonization or infection. Although some studies have focused on modification of the airway microenvironment to restore function to previously functional tissue, a composition disclosed herein is capable of activating mucociliary clearance functions in epithelial cells otherwise devoid of such functions.

Ascorbic acid can directly modulate CFTR activity (Proc Natl Acad Sci USA. 2004 Mar. 9; 101(10): 3691-3696.). However, ascorbic acid has also been shown to have limited effect on mucociliary clearance (BMC Complement Altern Med. 2013; 13: 110.) and limited clinical efficacy (Sakasura Ann Otol 82, 1973; Adewale, A. T. et al., American Journal of Respiratory Cell and Molecular Biology 63, 362-373 (2020)). However, a composition disclosed herein can restore mucociliary function in the absence of a bacterial infection and regardless of CFTR presence or dysfunction.

When administered individually, glutathione, ascorbic acid, and bicarbonate do not have any clinical impact on mucociliary clearance or clinical outcomes (Sakasura Ann Otol 82; 1973; Am J Respir Crit Care Med. 2013 Jul. 1; 188(1):83-9. doi: 10.1 164/rccm.201303-04270C.). Moreover, the effects of bicarbonate have been inconclusive despite clinical data that directly effects the pH of the airway (Adewale, A. T. et al., American Journal of Respiratory Cell and Molecular Biology 63, 362-373 (2020); and Gomez, C. C. S., et al., Clin Drug Investig. 2020 February;40(2):105-117. doi: 10.1007/s40261-019-00861-x. PMID: 31721070).

In some aspects, a composition disclosed herein can generate a synergistic effect between glutathione, ascorbic acid, and bicarbonate, and can activate mucociliary clearance to levels seen in normal cells. In some aspects, composition disclosed herein are useful in upregulating mucociliary clearance in a subject suffering from or at risk of impaired mucociliary clearance.

In some aspects, the disclosure comprises a method of upregulating mucociliary clearance in a subject suffering from or at risk of impaired mucociliary clearance comprising administering to the subject a composition comprising: (a) glutathione, a glutathione derivative, a glutathione conjugate, a pharmaceutically acceptable salt thereof and (b) an organic acid.

In another aspect, the organic acid is ascorbic acid. In another aspect, the composition further comprises: (c) a bicarbonate salt. In another aspect, the bicarbonate salt is sodium bicarbonate or potassium bicarbonate. In some aspects, the organic acid in is ascorbic acid. In some aspects, the composition comprises: (a) glutathione; (b) ascorbic acid; and (c) sodium bicarbonate. In certain aspects, the molar ratio of (a):(b):(c) is about 0.1-0.5:0.5-1:1 (e.g., about 0.49: about 0.50: about 1). In other aspects, the molar ratio of (a):(b):(c) is about 0.1-0.5:1: 1 (e.g., about 0.49: about 1: about 1).

In another aspect, the amount of each of component the glutathione conjugate and/or the glutathione, or a pharmaceutically acceptable salt thereof, the organic acid, or a pharmaceutically acceptable salt thereof, and the bicarbonate salt of the composition is present such that the amount of bicarbonate salt results in a pH in a range from about 5.5 to about 14. In some aspects, the pH of a composition disclosed herein is from about 5.5 to about 14 (e.g. 5.5 to 7.5). In some aspects, the pH of a composition disclosed herein is from about 6 to about 14 (e.g., 6 to 7.5). In some aspects, the pH of the composition is 7±1.5, 7±1.4, 7±1.3, 7±1.2, 7±1.1, 6±0.5, 6±0.4, 6±0.3, 6±0.2, 6±0.5, 6±0.1, or about 6.

In another aspect, the composition further comprises from about 0.01% to about 5% by weight of a pharmaceutically acceptable thiocyanate salt.

In another aspect, the composition is in the form of a particle. In another aspect, the particle is mixed with a gas or liquid propellant for use in an inhalation therapy. In some aspects, the inhalation therapy comprises administration of a nebulized formulation. In some aspects, the inhalation therapy comprises administration of a dry powder formulation.

In another aspect, the glutathione is reduced glutathione.

In another aspect, administration of the composition to the subject gives a concentration of about 0.1 mM to about 1.0 mM glutathione in the airway surface liquid of the subject. In another aspect, administration of the composition to the subject gives a concentration of about 0.5 mM to about 3.0 mM thiocyanate in the airway surface liquid of the subject.

In some aspects, the oxidized glutathione (e.g., % GSSG) in a composition disclosed herein is less than about 20%, less than about 18%, less than about 16%, less than about 15%, less than about 12%, less than about 10%, less than about 9%, less than about 8%, less than about 7%, less than about 6%, less than about 5%, less than about 4%, or less than about 3% by weight of the total glutathione in a composition disclosed herein after storage of a composition disclosed herein for 4 weeks (e.g., at 5° C. in a N₂ atmosphere and/or ambient atmosphere).

In some aspects, the reduced glutathione in a composition disclosed herein is more than about 80%, more than about 82%, more than about 84%, more than about 85%, more than about 88%, more than about 90%, more than about 91%, more than about 92%, more than about 93%, more than about 94%, more than about 95%, more than about 96%, or more than about 97% by weight of the total glutathione in a composition disclosed herein after storage of a composition disclosed herein for 4 weeks at about 5° C. (e.g., in a N₂ or ambient atmosphere).

In some aspects, the reduced ascorbic acid (e.g., % ASC) is more than about 80%, more than about 85%, more than about 86%, more than about 87%, more than about 88%, more than about 89%, or more than about 90% by weight of the ascorbic acid in a composition disclosed herein after storage of a composition disclosed herein for 4 weeks (e.g., at 5° C. in a N₂ atmosphere and/or ambient atmosphere).

In some aspects, the oxidized ascorbic acid in a composition disclosed herein is less than about 20%, less than about 18%, less than about 16%, less than about 15%, less than about 12%, less than about 10%, or less than about 9% by weight of the total ascorbic acid in a composition disclosed herein after storage of a composition disclosed herein for 4 weeks (e.g., at 5° C. in a N₂ atmosphere and/or ambient atmosphere).

In some aspects, the composition is an aqueous solution or a dry powder. In some aspects, the composition is administered by inhalation to the subject.

In some aspects, administering the composition decreases mucus viscosity of the patient. In some aspects, administering the composition increases ciliary beat frequency of the patient's airway epithelial cells. In some aspects, administering the composition increases the mucociliary transport rate of the patient's airway epithelial cells. In some aspects, administering the composition increases the number of the cilia in the airway of the subject. In some aspects, administering the composition increases the length of the cilia in the airway of the subject. In some aspects, administering the composition improves the function of the cilia in the airway of the subject. In some aspects, administering said composition increases the airway surface liquid height of the patient.

In some aspects, the patient having one or more COVID symptoms and/or infection with SARS-CoV-2 suffers from or is at risk of suffering from a chronic inflammatory airway disease. In some aspects, the inflammatory airway disease is cystic fibrosis, bronchiectasis (e.g., non-cystic fibrosis bronchiectasis or cystic fibrosis bronchiectasis), asthma, chronic obstructive pulmonary disease, or pulmonary fibrosis. In some aspects, the inflammatory airway disease is cystic fibrosis. In some aspects, the airway epithelial cells of the patient lack mucociliary clearance ability prior to administration of said composition. In some aspects, the airway epithelial cells of said patient lack mucociliary clearance ability due to a genetic deficiency.

In some aspects, the patient having one or more COVID symptoms and/or infection with SARS-CoV-2 has received a lung transplant.

In some aspects, the airway epithelium of the patient is not colonized by bacteria. In other aspects, the airway epithelium of the patient is colonized by bacteria. In some aspects, the patient suffers from an active bacterial infection. In other aspects, the patient does not suffer from an active bacterial infection. In some aspects, the patient suffers from a recurrent bacterial infection. In some aspects, the patient suffers from a refractory bacterial infection.

In some aspects, the patient is a pediatric patient. In other aspects, the patient is an adult patient.

In some aspects, the patient suffers from an active SARS-CoV-2 infection, e.g., tests positive for COVID. In some aspects, the subject is suffering from long COVID. In some aspects, the subject is suffering from inflammation incident to a SARS-CoV-2 infection. In some aspects, the subject is dependent on supplemental oxygen.

In some aspects, administering the composition upregulates mucus clearance without resulting in neutrophilia.

Certain aspects of the disclosure are directed to reducing inflammation in the lungs of a subject suffering from a SARS-CoV-2 infection. Hallmarks of inflammation in airway disease include the production of neutrophil extracellular traps (NETs), increased neutrophil myeloperoxidase activity, increased nitric oxide production, and increased production of pro-inflammatory cytokines. Studies suggest that ascorbic acid specifically decreases myeloperoxidase (MPO) activity and promotes leukocyte recruitment, and that the presence of MPO is directly associated with clinical complication and deterioration (Free Radic Biol Med. 2017 Oct. 2; 113:236-243). Moreover, neutrophil extracellular trap formation caused by phorbol myristate acetate (PMA) is prevented under acidic conditions and elicited under more basic conditions resulting from high concentrations of sodium bicarbonate.

In some aspects, a composition disclosed herein directly inhibits the inflammatory process. In some aspects, a composition disclosed herein inhibits MPO activity, as well as downregulates cytokines associated with NET formation, macrophage activation and neutrophil and T cell recruitment in a mechanism independent of reducing functions. In some aspects, a composition disclosed herein can downregulate the level of pathological nitric oxide production and therefore not disrupt endogenous antibacterial properties. In some aspects, administering said composition reduces the patient's fractional exhaled nitric oxide (FeNO) by at least 20%. Accordingly, in some aspects, a composition disclosed herein is useful for reducing airway inflammation in a subject suffering from or at risk of airway inflammation.

In some aspects, the disclosure comprises a method of reducing airway inflammation in a subject having one or more COVID symptoms and/or infection with SARS-CoV-2 suffering from or at risk of airway inflammation comprising administering to the subject a composition comprising: (a) glutathione, a glutathione derivative, a glutathione conjugate, a pharmaceutically acceptable salt thereof and (b) an organic acid.

In another aspect, the organic acid is ascorbic acid. In another aspect, the composition further comprises: (c) a bicarbonate salt. In another aspect, the bicarbonate salt is sodium bicarbonate or potassium bicarbonate. In some aspects, the composition comprises: (a) glutathione; (b) ascorbic acid; and (c) sodium bicarbonate. In certain aspects, the molar ratio of (a):(b):(c) is about 0.1-0.5:0.5-1:1 (e.g., about 0.49: about 0.50: about 1). In other aspects, the molar ratio of (a):(b):(c) is about 0.1-0.5:1: 1 (e.g., about 0.49: about 1: about 1).

In another aspect, the amount of each of component the glutathione conjugate and/or the glutathione, or a pharmaceutically acceptable salt thereof, the organic acid, or a pharmaceutically acceptable salt thereof, and the bicarbonate salt of the composition is present such that the amount of bicarbonate salt results in a pH in a range from about 5.5 to about 14. In some aspects, the pH of a composition disclosed herein is from about 5.5 to about 14 (e.g. 5.5 to 7.5). In some aspects, the pH of a composition disclosed herein is from about 6 to about 14 (e.g., 6 to 7.5). In some aspects, the pH of the composition is 7±1.5, 7±1.4, 7±1.3, 7±1.2, 7±1.1, 6±0.5, 6±0.4, 6±0.3, 6±0.2, 6±0.5, 6±0.1, or about 6.

In another aspect, the composition further comprises from about 0.01% to about 5% by weight of a pharmaceutically acceptable thiocyanate salt.

In another aspect, the composition is in the form of a particle. In another aspect, the particle is mixed with a gas or liquid propellant for use in an inhalation therapy. In some aspects, the inhalation therapy comprises administration of a nebulized formulation. In some aspects, the inhalation therapy comprises administration of a dry powder formulation.

In another aspect, the glutathione is reduced glutathione.

In another aspect, administration of the composition to the subject gives a concentration of about 0.1 mM to about 1.0 mM glutathione in the airway surface liquid of the subject. In another aspect, administration of the composition to the subject gives a concentration of about 0.5 mM to about 3.0 mM thiocyanate in the airway surface liquid of the subject.

In some aspects, the oxidized glutathione (e.g., % GSSG) in a composition disclosed herein is less than about 20%, less than about 18%, less than about 16%, less than about 15%, less than about 12%, less than about 10%, less than about 9%, less than about 8%, less than about 7%, less than about 6%, less than about 5%, less than about 4%, or less than about 3% by weight of the total glutathione in a composition disclosed herein after storage of a composition disclosed herein for 4 weeks (e.g., at 5° C. in a N₂ atmosphere and/or ambient atmosphere).

In some aspects, the reduced glutathione in a composition disclosed herein is more than about 80%, more than about 82%, more than about 84%, more than about 85%, more than about 88%, more than about 90%, more than about 91%, more than about 92%, more than about 93%, more than about 94%, more than about 95%, more than about 96%, or more than about 97% by weight of the total glutathione in a composition disclosed herein after storage of a composition disclosed herein for 4 weeks at about 5° C. (e.g., in a N₂ or ambient atmosphere).

In some aspects, the reduced ascorbic acid (e.g., % ASC) is more than about 80%, more than about 85%, more than about 86%, more than about 87%, more than about 88%, more than about 89%, or more than about 90% by weight of the ascorbic acid in a composition disclosed herein after storage of a composition disclosed herein for 4 weeks (e.g., at 5° C. in a N₂ atmosphere and/or ambient atmosphere).

In some aspects, the oxidized ascorbic acid in a composition disclosed herein is less than about 20%, less than about 18%, less than about 16%, less than about 15%, less than about 12%, less than about 10%, or less than about 9% by weight of the total ascorbic acid in a composition disclosed herein after storage of a composition disclosed herein for 4 weeks (e.g., at 5° C. in a N₂ atmosphere and/or ambient atmosphere).

In some aspects, the composition is an aqueous solution or a dry powder. In another aspect, the composition is administered by inhalation to the subject.

In some aspects, administering the composition inhibits myeloperoxidase activity of the patient's neutrophils. In some aspects, administering the composition decreases the formation of neutrophil extracellular traps. In some aspects, administering the composition downregulates the production of nitric oxide from the patient's neutrophils. In some aspects, administering the composition reduces the patient's fractional exhaled nitric oxide by at least 20%, at least 21%, at least 22%, at least 23%, at least 24%, or at least 25%. In some aspects, administering the composition reduces the patient's baseline fractional exhaled nitric oxide by at least 20%.

In some aspects, administering the composition downregulates the production of at least one inflammatory cytokine. In some aspects, the at least one inflammatory cytokine comprises a cytokine associated with macrophage activation and/or neutrophil and T cell recruitment. In some aspects, the at least one inflammatory cytokine comprises TNF-α. In some aspects, the at least one inflammatory cytokine comprises IL-6. In some aspects, the at least one inflammatory cytokine comprises IL-8. In some aspects, the at least one inflammatory cytokine comprises MIP-la. In some aspects, the at least one inflammatory cytokine comprises MIP-lb. In some aspects, the at least one inflammatory cytokine comprises MMP-9.

In some aspects, the patient suffers from or is at risk of suffering from a chronic inflammatory airway disease. In some aspects, the inflammatory airway disease is cystic fibrosis, bronchiectasis (e.g., non-cystic fibrosis bronchiectasis or cystic fibrosis bronchiectasis), asthma, chronic obstructive pulmonary disease, or pulmonary fibrosis. In some aspects, the inflammatory airway disease is cystic fibrosis. In some aspects, the patient suffers from a SARS-CoV-2 infection. In some aspects, the subject is suffering from long COVID. In some aspects, the subject is suffering from inflammation incident to a SARS-CoV-2 infection. In some aspects, the subject is oxygen dependent.

In some aspects, the patient having one or more COVID symptoms and/or infection with SARS-CoV-2 has received a lung transplant.

In some aspects, the airway epithelium of the patient having one or more COVID symptoms and/or infection with SARS-CoV-2 is not colonized by bacteria. In some aspects, the airway epithelium of the patient having one or more COVID symptoms and/or infection with SARS-CoV-2 is colonized by bacteria. In certain aspects, the patient having one or more COVID symptoms and/or infection with SARS-CoV-2 suffers from an active bacterial infection. In other aspects, the patient having one or more COVID symptoms and/or infection with SARS-CoV-2 does not suffer from an active bacterial infection. In some aspects, the patient having one or more COVID symptoms and/or infection with SARS-CoV-2 suffers from a recurrent bacterial infection. In some aspects, the patient having one or more COVID symptoms and/or infection with SARS-CoV-2 suffers from a refractory bacterial infection.

In some aspects, the patient having one or more COVID symptoms and/or infection with SARS-CoV-2 is a pediatric patient. In other aspects, the patient is an adult patient.

In some aspects, administering the composition does not significantly alter the pH of the patient's airway epithelia.

### Administration

The therapeutic methods of this disclosure can be accomplished by administering (e.g., to the airway of a subject) a composition disclosed herein to a subject having one or more COVID symptoms and/or infection with SARS-CoV-2. Administration of a composition disclosed herein can be performed before, during, or after the onset of the disease, condition, or disorder of interest. Typically, the pharmaceutical compositions are sterile, and contain no toxic, carcinogenic, or mutagenic compounds that would cause an adverse reaction when administered to the subject.

In some aspects, a composition disclosed herein is administered in a manner compatible with the dosage formulation in such an amount as will be effective for the desired result. In some aspects, a composition disclosed herein is administered to the subject in a therapeutically effective amount. A therapeutically effective amount of a composition disclosed herein required for use in therapy varies with the nature of the condition being treated, the length of time that activity is desired, and the age and the condition of the patient, and ultimately is determined by the attendant physician. Dosage amounts and intervals can be adjusted individually to provide plasma levels of glutathione that are sufficient to maintain the desired therapeutic effects. The desired dose can be administered in a single dose, or as multiple doses administered at appropriate intervals, for example as one, two, three, four or more subdoses per day. Multiple doses often are desired, or required. For example, glutathione, a glutathione derivative, a glutathione conjugate, a pharmaceutically acceptable salt thereof, or any combination thereof can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days' rest, and another five daily doses (5/2/5); or, any dose regimen determined to be appropriate for the circumstance.

In some aspects, a composition disclosed herein can be administered for a sustained period, such as for at least about one month, at least about 2 months, at least about 3 months, at least about 6 months, or at least about 12 months or longer (e.g., as a chronic life-long treatment).

Any suitable dosing schedule can be followed. For example, the dosing frequency can be a once weekly dosing. The dosing frequency can be a once daily or multiple times daily dosing. The dosing frequency can be more than once weekly dosing. The dosing frequency can be more than once daily dosing, such as any one of 2, 3, 4, 5, 6, 7, 8, 9, 10 or more than 10 daily doses. The dosing frequency can be intermittent (e.g., multiple daily dosing for 7 days followed by no doses for 7 days, repeated for any 14 day time period, such as 2 months, 4 months, 6 months or more). The dosing frequency can be continuous (e.g., one weekly dosing for continuous weeks).

Glutathione, a glutathione derivative, a glutathione conjugate, a pharmaceutically acceptable salt thereof, or any combination thereof used in a therapeutic method of the present disclosure can be administered in an amount of about 0.005 to about 1,000 milligrams per dose, about 0.05 to about 250 milligrams per dose, or about 0.5 to about 100 milligrams per dose. For example, glutathione, a glutathione derivative, a glutathione conjugate, a pharmaceutically acceptable salt thereof, or any combination thereof can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 1,000 milligrams.

The dosage of glutathione, a glutathione derivative, a glutathione conjugate, a pharmaceutically acceptable salt thereof, or any combination thereof, or a composition containing the same, can be from about 1 ng/kg to about 200 mg/kg, about 1 g/kg to about 100 mg/kg, or about 1 mg/kg to about 50 mg/kg. The dosage of a composition can be at any dosage including, but not limited to, about 1 g/kg. The dosage of a composition can be at any dosage including, but not limited to, about 1 g/kg, about 10 g/kg, about 25 g/kg, about 50 ag/kg, about 75 ag/kg, about 100 ag/kg, about 125 ag/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual patient, which can vary with the age, weight, and response of the particular patient.

In one aspect, glutathione, a glutathione derivative, a glutathione conjugate, a pharmaceutically acceptable salt thereof, or any combination thereof is delivered to the upper third of the nasal cavity, to the superior meatus, the olfactory region and/or the sinus region of the nose. The olfactory region is a small area that is typically about 2-10 cm² in man located in the upper third of the nasal cavity for deposition and absorption by the olfactory epithelium and subsequent transport by olfactory receptor neurons. Located on the roof of the nasal cavity, in the superior meatus, the olfactory region is useful for delivery in some aspects, because it is the only known part of the body in which an extension of the CNS comes into contact with the environment (Bois et al. Fundamentals of Otolaryngology, p. 184, W. B. Saunders Co., Philadelphia, 1989).

In some aspects, a composition disclosed herein can be administered in a single “shock” dose, for example, during a bronchoscopy. In other aspects, the methods of the disclosure can be carried out on an as-needed basis by self-medication.

Any of the dosing frequencies can be used with any dosage amount. Further, any of the dosing frequencies and/or dosage amounts can be used with a composition disclosed herein.

A composition disclosed herein can be delivered in any suitable volume of administration, In representative aspects of the disclosure, the administration volume for intranasal delivery ranges from about 25 microliters to 200 microliters or from about 50 to 150 microliters or from about 50, 100, 250 or 500 microliters to about 1, 2, 3, 3.5 or 4 milliliters in a human. Typically, the administration volume is selected to be large enough to allow for delivery of therapeutic quantities while accounting for dilution in ASL in maintenance conditions in relatively normal airways and in cystic fibrosis (CF) airways.

In some aspects, a composition disclosed herein can find use in both veterinary and/or medical applications. Suitable subjects of the present disclosure include, but are not limited to mammals. The term “mammal” as used herein includes, but is not limited to, primates (e.g., simians and humans), non-human primates (e.g., monkeys, baboons, chimpanzees, gorillas), bovines, ovines, caprines, ungulates, porcines, equines, felines, canines, lagomorphs, pinnipeds, rodents (e.g., rats, hamsters, and mice), etc. In some aspects of the present disclosure, the subject is a human. Human subjects include both males and females and subjects of all ages including neonatal, infant, juvenile, adolescent, adult, and geriatric subjects.

In some aspects, a composition disclosed herein is administered one or more times daily (e.g., 1, 2, 3, 4, 5, 6, 7, 8, 9, 10 or more times a day). In particular aspects, the subject is a human.

In some aspects of the methods of this disclosure, a composition disclosed herein can be administered via inhalation, intranasally, via the eye, via the ear, via sinus irrigation, or via bronchoscope, or any combination thereof.

Intranasal administration of a composition disclosed herein can be achieved by any known method. In particular aspects, intranasal administration is by inhalation (e.g., using an inhaler, atomizer or nebulizer device), alternatively, by spray, tube, catheter, syringe, dropper, packtail, pipette, pledget, and the like.

In some aspects, administration of a composition disclosed herein comprises intra-sinus administration by nebulizer. In some aspects, administration of a composition disclosed herein comprises instillation during sinus surgery.

In some aspects, the method of delivery is by nasal drops, spray or aerosol. As used herein, aerosols can be used to deliver powders, liquids or dispersions (solids in liquid).

In some aspects, the pharmaceutical formulation is directed upward during administration, so as to enhance delivery to the upper third (e.g., the olfactory epithelium in the olfactory region) and the side walls (e.g., nasal epithelium) of the nasal cavity. Further, orienting the subject's head in a tipped-back position or orienting the subject's body in Mygind's position or the praying-to-Mecca position can be used to facilitate delivery to the olfactory region.

The formulations can be provided in single or multidose form. In the latter case a means of dose metering can be provided. In the case of a dropper or pipette, this can be achieved by the patient or caregiver administering an appropriate, predetermined volume of the composition. In the case of a spray, this can be achieved, for example, by means of a metering atomizing spray pump.

In a further aspect, the present disclosure provides an intranasal spray device comprising a composition disclosed herein.

Many devices are known in the art for nasal delivery. Exemplary devices include particle dispersion devices, bidirectional devices, and devices that use chip-based ink jet technologies.

A composition disclosed herein can be present, for example, as a solid formulation, such as a particle formulation, or as a solution. When in a particle formulation, the particles can be mixed with gases, or liquid propellants, for use in an inhalation therapy. In some aspects, the inhalation therapy comprises administration of a nebulized formulation. In some aspects, the inhalation therapy comprises administration of a dry powder formulation. Other solid formulations include formulations for oral administration, buccal administration or colonic administration, and suppositories for rectal or vaginal administration. Exemplary formulations include, but are not limited to, the following: eye drops, nebulizers, topical gels and ointments, dry powders, particles, sprays, liquids, anesthetic machines or vaporizers, autoinjectors, intrauterine devices, respimats, liniments, liposomes, lotions, formulations for intramuscular, intrathecal, or subcutaneous injection, douches, infusions, and face masks.

In solution form, the formulations can be in the form of sprays for intranasal administration, formulations for use in nebulizers, and formulations for rectal administration, such as enemas and colonies. Solutions that include water-miscible organic solvents, such as propylene glycol and/or glycerol, and other components normally found in vaginal and rectal lubricants, can also be used. Regardless of the solvents used, the solvent is typically present in a weight ratio of from about 15 to about 85 percent by weight, relative to the weight of the solids, and, more typically, is from about 50 to about 85% by weight.

The compositions and/or formulations of this invention can be used to treat disorders associated with a mucosal membrane, by delivering the compositions and/or formulations to the mucosal membrane(s) to be treated. In some aspects, the mucosal membrane can be in or near the lungs, such as the deep lung (alveolar region), and in other aspects, the mucosal membrane(s) can be in or near one or more of the eyes, mouth, nose, rectum, and/or vagina.

### Additional Therapeutic Agents

In another aspect, the present disclosure provides a method comprising administering to the subject an effective amount of an additional therapeutic agent. In some aspects, the additional therapeutic agent is an antibody. In some aspects, the additional therapeutic agent is an antiviral. In some aspects, the antiviral is nirmatrelvir/ritonavir (Paxlovid) or molnupiravir (Lagevrio). In some aspects, the additional therapeutic agent is a systemic or inhaled corticosteroid. In some aspects, the additional therapeutic agent is an antibiotic. In some aspects, the antibiotic is an antibiotic disclosed herein. In some aspects, the antibiotic is one or more of azithromycin, bactrim, levoquin, azithromycin, cipro, linezolid, or vancomycin. In some aspects, the additional therapeutic agent is an immunosuppression agent. In some aspects, the immunosuppression agent is an anti-IL8 therapy. In some aspects, the immunosuppression agent is tocilizumab.

Drugs administered to the lungs are often associated with certain side effects, in some cases because of dosage, and in other cases because they damage the lung tissue. In some aspects, therapeutic agents combined with the compositions disclosed herein are effective at lower doses, and at such lower doses, the incidence of side effects can be reduced. In other aspects, where the therapeutic agent interacts unfavorably with lung tissue, the compositions described herein can help to restore homeostasis to the lung tissue, and thus help minimize or eliminate damage caused by the therapeutic agents.

In one aspect, the therapeutic agent is selected from the group consisting of Fluticasone, Budesonide, Mometasone, Ciclesonide, Flunisolide, Beclomethasone, Albuterol, Levalbuterol, Ipratropium, Tiotropium, Formoterol, Arformoterol, Indacaterol, Aclidinium, Cayston, Pirbuterol, corticosteroids, and any combination thereof.

Additional therapeutic agents that can be combined with the compositions and formulations of this invention include, but are not limited to, Fluticasone (for example, sold as Flovent diskus 50 or as Flonase, GlaxoSmithKline), Budesonide (for example, sold as Pulmicort respules or Rhinocort by Astra Zeneca (“AZ”), Mometasone (sold as Nasonex as a spray, or as Asmanex Twisthaler by Merck/S-P), Ciclesonide (sold as Alvesco or Onmaris by Takeda Pharmaceuticals), Flunisolide (sold as Aerobid by Roche Palo or by Aerospan HFA by GSK), Beclomethasone (sold as Qvar or Onasl by Teva Pharmaceuticals), Albuterol (sold as ProAir HFA by Teva and as Ventolin HFA by GSK), Levalbuterol (sold as Xopenex by Sunovion), Ipratropium (sold as Atrovent by BI), Tiotropium (sold as Spiriva by BI), Salmeterol (sold as Serevent by GSK), Formoterol (sold as Foradil by Novartis and as Perforomist by Dey Pharma), Arformoterol (sold as Brovana by Sunovion), Indacaterol (sold as Arcapta by Novartis), Aclidinium (sold as Tudorza by Forest Labs), Pirbuterol (sold as Maxair by Medicis), and any combination thereof.

The present methods encompass administering one or more additional therapeutic agents to the subject in combination with a composition disclosed herein. In one aspect, a composition disclosed herein further comprises, or the composition is administered in combination or in alternation with, an additional therapeutic agent. That is, in some aspects, the composition and further therapeutic agents are directed to the same locus in the same formulation, and in other aspects, the composition can be administered via one pathway, and the further therapeutic agent(s) can be administered via a different pathway.

The additional therapeutic agent is typically selected from drugs known as useful in treating the disease, condition, or disorder afflicting the subject in need thereof. The choice of additional therapeutic agent(s) will depend on the disease, condition, or disorder to be treated or prevented in a subject. This determination is within the capability of those skilled in the art, especially in light of the present disclosure.

In one aspect, one additional therapeutic agent is administered to the subject. In another aspect, two additional therapeutic agents are administered to the subject. In another aspect, three additional therapeutic agents are administered to the subject. In another aspect, four additional therapeutic agents are administered to the subject. In another aspect, five additional therapeutic agents are administered to the subject. In another aspect, five or more additional therapeutic agents are administered to the subject. Non-limiting exemplary therapeutic agents are antifungal agents, antiviral agents, antibacterial agents, anti-inflammatory agents, immunosuppressive agents, corticosteroids, bronchodilators, airway modulators, alpha lipoic acid, alpha tocopherol, docosahexanic acid, proline, glycine, curcumin, arginine, thiocyanate, glutathione, oxidized glutathione, reduced glutathione, cysteine, hypothiocyanate, lactoferrin, and lactoperoxidase, and any combination thereof. In another aspect, the one or more therapeutic agents are glutathione, oxidized glutathione, or reduced glutathione.

A composition disclosed herein and an additional therapeutic agent can be administered together as a single-unit dose or separately as multi-unit doses, wherein a composition disclosed herein is administered before the additional therapeutic agent, after the additional therapeutic agent, or concurrently with the additional therapeutic agent. One or more doses of a composition disclosed herein and/or one or more doses of the additional therapeutic agent can be administered to the subject.

In another aspect, the additional therapeutic agent treats the desired disorder for which it is administered, but can cause certain side effects, e.g., drying of the mucosal membranes that results in discomfort and/or injury that can be addressed by administering a composition disclosed herein.

In another aspect, a composition disclosed herein treats the desired disorder for which it is administered, but can cause certain side effects, e.g., drying of the mucosal membranes that results in discomfort and/or injury that can be addressed by administering an additional therapeutic agent.

In some aspects, the one or more additional therapeutic agent(s) and a composition disclosed herein both treat the underlying disorder, though via different means, such that an additive or synergistic effect can be achieved. As a result, in some aspects of this aspect, lower doses of the additional therapeutic agent can be effective, which lower doses can result in fewer side effects, or provide other benefits to the subject.

In another aspect, the additional therapeutic agent is an inhaled corticosteroid (ICS) or bronchodilator.

### Prophylaxis

In addition to providing methods of treatment, a composition disclosed herein can be used to reduce the symptoms of or provide prevention of various diseases and disorders associated with SARS-CoV-2 infection, impaired mucociliary clearance, and/or airway inflammation. In some aspects, a composition disclosed herein can be used to reduce the symptoms of or prevent SARS-CoV-2 infection or the symptoms associated therewith.

Aspects of the present disclosure can be further defined by reference to the following non-limiting examples, which describe in detail preparation of certain compositions of the present disclosure and methods for using compositions of the present disclosure. It will be apparent to those skilled in the art that many modifications, both to materials and methods, can be practiced without departing from the scope of the present disclosure.

### Exemplary Embodiments

Provided are exemplary, non-limiting embodiments of the present disclosure.

E1. A method of treating, preventing, or reducing the risk of an infection by SARS-CoV-2 in a subject in need thereof, comprising administering to the subject a therapeutically effective amount of a composition comprising:
(a) glutathione, a glutathione derivative, a glutathione conjugate, or a pharmaceutically acceptable salt of glutathione, a glutathione derivative, or a glutathione conjugate; and
(b) an organic acid or a pharmaceutically acceptable salt thereof.

E2. A method of reducing one or more symptoms or signs of an infection by SARS-CoV-2, comprising administering to the subject a therapeutically effective amount of a composition comprising:
(a) glutathione, a glutathione derivative, a glutathione conjugate, or a pharmaceutically acceptable salt of glutathione, a glutathione derivative, or a glutathione conjugate; and
(b) an organic acid or a pharmaceutically acceptable salt thereof.

E3. The method of E2, wherein the one or more symptoms or signs of infection by SARS-CoV-2 comprise lung inflammation, fever, chills, cough, shortness of breath, difficulty breathing, fatigue, muscle aches, body aches, headache, reduction in ability to taste, reduction in ability to smell, sore throat, congestion, runny nose, nausea, vomiting, diarrhea, chest pain, confusion, inability to wake, inability to stay awake, discolored skin, discolored lips, or discolored nail beds.

E4. A method of protecting the lungs of a subject suffering from SARS-CoV-2 from cilia damage comprising administering to the airway of the subject a composition comprising: (a) glutathione, a glutathione derivative, a glutathione conjugate, or a pharmaceutically acceptable salt of glutathione, a glutathione derivative, or a glutathione conjugate; and (b) an organic acid or a pharmaceutically acceptable salt thereof.

E5. The method of E4, wherein the cilia damage comprises cilia shortening.

E6. The method of any one of E1-E5, wherein the molar ratio of (a):(b) is about 0.1-0.5:0.5-1.

E7. The method of any one of E1-E5, wherein the composition further comprises (c) bicarbonate or a pharmaceutically acceptable salt thereof.

E8. The method of E7, wherein the composition comprises sodium bicarbonate or calcium bicarbonate.

E9. The method of E7 or E8, wherein the molar ratio of (a):(b):(c) is about 0.1-0.6:0.5-1:1.

E10. The method of any one of E1-E9, wherein SARS-CoV-2 is a variant is selected from the group consisting of an Alpha variant (e.g., B.1.1.7 and Q lineages), a Beta variant (e.g., B.1.351 and descendent lineages), a Gamma variant (e.g., P.1 and descendent lineages), a Delta variant (e.g., B.1.617.2 and AY lineages), an Epsilon variant (e.g., B.1.427 and B.1.429), an Eta variant (e.g., B.1.525), an Iota variant (e.g., B.1.526), a Kappa variant (e.g., B.1.617.1), 1.617.3, a Mu variant (e.g., B.1.621, B.1.621.1), a Zeta variant (e.g., P.2), or an Omicron variant (e.g., B.1.1.529, BA.1, BA.1.1, BA.2, BA.3, BA.4 and BA.5 lineages).

E11. The method of any one of E1-E8, wherein the composition comprises glutathione.

E12. The method of any one of E-E1, wherein the organic acid is ascorbic acid or a pharmaceutically acceptable salt thereof.

E13. The method of any one of E1-E12 wherein the composition is administered to the lungs by an inhalable dosage form.

E14. The method of E13, wherein the inhalable dosage form is a metered dose inhaler, a dry powder inhaler, or a nebulizer.

E15. The method of any one of E1-E14 wherein the subject has long COVID.

E16. The method of any one of E1-E15, wherein the subject has a chronic airway disease or condition.

E17. The method of any one of E1-E16, wherein the subject belongs to a subject population having an increased risk of SARS-CoV-2 infection, or an increased risk of a severe infection by SARS-CoV-2.

E18. The method of any one of E1-E17, wherein the subject is aged 65 or greater.

E19. The method of any one of E1-E18, wherein the subject suffers from an acute SARS-CoV-2 infection.

E20. The method of any one of E1-E19, wherein the subject has developed one or more SARS-CoV-2-related sequelae.

E21. The method of E20, wherein the one or more SARS-CoV-2-related sequelae comprises a bronchiectasis.

E22. The method of E21, wherein the bronchiectasis is traction bronchiectasis.

E22. The method of E21, wherein the bronchiectasis is non-cystic fibrosis bronchiectasis.

E23. The method of E20, wherein the one or more SARS-CoV-2-related sequelae is characterized by a symptom selected from one or more of ageusia, myalgia, arthralgia, parosmia, anosmia, fatigue, headache, cough, chills, shivers, fever, dyspnea, sore throat, rhinorrhea, diarrhea, brain fog, nausea, subjective fever, abdominal pain, vomiting, rash, skin abnormality, and blood clots.

E24. The method of any one of E1-E23, wherein the composition blocks SARS-CoV-2 replication (e.g., in the cells of the subject's airway).

E25. The method of any one of E1-E24, wherein the composition according to the methods disclosed herein reduces SARS-CoV-2 viral load (e.g., in the cells of the subject's airway).

E26. The method of any one of E1-E25, wherein the composition does not decrease SARS-CoV-2 infectivity (e.g., in the cells of the subject's airway).

E27. The method of any one of E1-E26, wherein the method (i) increases the ciliary beat frequency of the patient's airway epithelial cells, (ii) increases the number of the cilia in the airway of the subject, and/or (iii) increases the length of the cilia in the airway of the subject.

It is understood that the examples and aspects described herein are for illustrative purposes only and that various modifications or changes in light thereof will be suggested to persons skilled in the art and are to be included within the spirit and purview of this application.

### EXAMPLES

### Example 1: Mucociliary Transport (MCT) Activation Assay

A panel of agents was evaluated for their activity against SARS-CoV-2 in vitro. To assess the antiviral activity of these drugs, terminally differentiated human bronchial epithelial cells (HBECs) were grown in an air-liquid interface (ALI), as a model of SARS-CoV-2 infection. ALI cultures closely recapitulate key characteristics of the in vivo airway. Therefore, this model can be used to interrogate the mechanisms of tissue damage and cell death, as well as aspects of host response to infection (Liu, X., Wu, Y. & Rong, L., Virol Sin 35, 280-289 (2020); and Rijsbergen, L. C., van Dijk, L. L. A., Engel, M. F. M., de Vries, R. D. & de Swart, R. L. In Vitro Modelling of Respiratory Virus Infections in Human Airway Epithelial Cells—A Systematic Review. Front Immunol 12, 683002 (2021)). Furthermore, human airway cultures grown in an ALI system express angiotensin-converting enzyme 2 (ACE2), the host cell receptor of SARS-CoV2, as well as the serine protease TMPRSS2, required for the viral glycoprotein (protein S) priming, providing a physiologically relevant model for investigating anti-COVID-19 interventions (Hao, S. et al., mBio 11, 1-17 (2020); Ryu, G. & Shin, H. W., Immune Network 21, 1-16 (2021); and Fiege, J. K. et al., PLOS Pathogens 17, e1009292 (2021)).

In short, differentiated HBECs grown at the ALI on transwells microporous filters are washed with PBS to remove mucus. Test compounds are added basolaterally (camostat, ivacaftor) or apically (PAAG, HA, ARINA-1). After one hour of incubation, SARS-CoV2 virus is added at an MOI of 10. Unattached viral particles are removed after an hour of incubation. At this point, the transwell data corresponding to the time zero (before viral replication) was collected. The remainder of the transwells are incubated for 72 h, with re-addition of the apical compounds (PAAG, HA, ARINA-1) at 24 and 48 h. Cells are collected from the filters and used for RNA purification (together with the time zero cells). Finally, the RNA collected is used to measure the viral load by RT-qPCR. A detailed non-limiting protocol is provided herein, which a skilled artisan will understand can be modified for evaluation of alternative test agents without departing from the scope of the disclosure.

### Procurement and Growth of Human Primary Airway Epithelial Cells.

Primary HBEC were derived from lung explants after written informed consent was obtained from donor subjects using methods described previously (van Goor, F. et al., Proc Natl Acad Sci USA 106, 18825-18830 (2009); and Rowe, S. M. et al., Pulm Pharmacol Ther 23, 268 (2010)). Briefly, tissues were debrided immediately after surgical resection, washed twice in Minimum Essential Media (MEM) with 0.5 mg/ml dithiothreitol (DTT) (Sigma-Aldrich, St. Louis, MO) and 25 U/ml DNAse I (Roche, Basel, Switzerland), and then placed in dissociation media containing MEM, 2.5 U/ml DNAse I, 100 mg/ml ceftazidime, 80 mg/ml tobramycin, 1.25 mg/ml amphotericin B, and 4.4 U/ml pronase (Sigma-Aldrich) for 24-36 h at 4° C. Loosened airway epithelial cells were then expanded using Bronchial Epithelial Growth Medium (BEGM) (LONZA, Basel, Switzerland) supplemented with an additional 10 nM all-trans-retinoic acid (Sigma-Aldrich) that was exchanged every 24 hours. Following expansion, first or second passage, cells at 80-90% confluency were dissociated and seeded at a density of 0.5×10⁵ on Costar® Transwell 24-well filter inserts (cat. #3470, Corning Inc.) after coating with NIH 3T3 fibroblast conditioned media. Cells were differentiated at ALI for at least 5 weeks using medium PneumaCult™/heparin/hydrocortisone (Stemcell™ Technologies) plus penicillin/streptomycin antibiotics (exchanged three times a week), before further use. Cells from five different donors were used in this study: WT128, WT148, WT152, WT158 and WT210 cell lines.

Nasal cells from two healthy and two PCD patients [patient 1 genotype: CCDC39 c.2586+1G>A (splice donor) heterozygous pathogenic and CCDC39 c.830_831del (p.Thr277Argfs*3) heterozygous pathogenic; patient 2: DNAI1 exon 5, c.370C>T (p.Arg124Cys) heterozygous pathogenic and intron 1, c.48+2dup heterozygous pathogenic] were obtained from nasal brushes after written informed consent was obtained from donors and grown as co-culture with 3T3 J2 cells. When the cells become 80-90% confluent the cells were detached using 0.05% trypsin. A total of 1.25×10⁵ cells were plated in FNC coated Costar® Transwell 24-well filter inserts (cat. #3470, Corning Inc.). After three days medium from the apical compartments was removed and differentiation medium (Pneumacult ALI maintenance medium) maintained at the basolateral side only. Medium was changed every other day until terminal differentiation (two and a half to three weeks) and then used for the experiments.

### Culture of 16HBE at the ALI.

16HBE cells (which express ACE-2 and TMPRSS2) were grown using Eagle's minimum essential medium (EMEM) (ATCC), supplemented with 10% fetal bovine serum (FBS) and were dissociated at 80-90% confluency and seeded at a density of 0.5×10⁵ on Costar® Transwell 24-well filter inserts (cat. #3470, Corning Inc.) after coating with FNC Coating Mix® (AthenaES® 0407H) and using EMEM medium. Upon reaching confluency, medium was removed from the apical side, and the cells were grown at the ALI for no longer than three weeks to avoid differentiation. They were then used in the antiviral assay described below.

Compounds Used in this Study and their Sources

The compounds used throughout this study were the following: ARINA-1; camostat mesylate (Millipore Sigma Cas #59721-29-8); poly-N(acetyl, arginyl) glucosamine (PAAG, donated); allopurinol (TCI, Cat. #A0907); ascorbic acid (LETCO Medical, Cat. #684471); reduced glutathione (Sigma, Cat. #G6013); sodium bicarbonate (LETCO Medical, Cat. #685940); N-acetylcysteine (EMID Millipore, Cat. #106425); L-; BAPTA/AM (EMD Millipore, Cat. #196419); sulforaphane (Medkoo Biosciences Inc. Cat. #202713250MG). Hydrosoluble and hydrophobic compounds were dissolved in sterile milli-Q water and dimethyl sulfoxide (DMSO), respectively. They were used at the indicated concentration depending on the experiment.

### HBEC/ALI-Based Antiviral Assay

All the antiviral assays performed using the following method were done using fully differentiated primary HBEC from several human donors, unless stated otherwise. For the antiviral assay the HBECs were washed two times to eliminate the mucus excess by adding 100 ul of phosphate buffered saline (PBS, 1×), incubating during 30 min at 37° C. under 5% CO₂, and aspirating the liquid without touching the cells. Subsequently, the vehicle (dimethyl sulfoxide (DMSO) or saline, depending on the compound used) and test compounds were added at the specified concentrations to the basolateral or apical side of the cell layers as desired. After incubation with the compounds for one hour, cells were exposed to SARS-CoV2 (Washington strain WA-1/USA ancestral clade A, obtained from BEI Resources, MOI=0.24, 10⁷ viral particles in 30 ul of medium) for an additional hour at 37° C. in a 5% CO₂ incubator. Each well contained an average of 1.7×10⁶ HBEC. Excess of unattached viral particles were aspirated from the filters and washed with 100 μl of PBS. Since this washing step removed the apical compounds, they were added again at this point (1 L).This was considered as the time zero of the experiment (t=0). At this point, control cells treated with vehicle and exposed to the virus for one hour were scraped from the transwell filters, collected in 100 ul of PBS and frozen at −80° C. for further analysis. This served as t=0 baseline virus controls, i.e., initial virus amount attached before replication. The rest of the filters, treated with either the vehicle or test compounds, were incubated at 37° C. under 5% CO₂ for 72 hours. During this time, compounds tested on the apical surface were added at 24 and 48 hours. That is, apical compounds were added four times: one hour before infecting the cells (which was washed out at the step of excess virus removal), then three times at 0, 24 and 48 h post infection (which were never removed from cell layer surface), unless stated otherwise. Compounds tested basolaterally were present in the medium for the duration of the experiment after viral exposure and washout. At the end of the 72-hour incubation period of the assay, cells were scraped and collected in 100 ul of PBS from each filter and frozen at −80° C. for further analysis of the viral load together with the baseline controls collected at t=0.

### Measurement of Viral Load by RT-qPCR

The effect of the drugs on blocking SARS-CoV2 infection of HBE was assessed by measuring the viral load through the quantification of the SARS-CoV2 genomic RNA copy number by RT-qPCR. Total RNA was purified from all the samples, including the baseline controls collected at t=0, using the QIAamp Viral RNA Mini Kit (Qiagen Cat. #52906). Purified RNA samples were diluted 1/50 and 5 ul of each dilution used to perform RT-qPCR to determine viral load (copy number). The RT-PCR was performed using the 2019-nCov CDC EUA kit (probe and oligos, IDT cat. #10006776) and Promega® GoTaq® 1 Step RT-PCR (Cat. #A6020) in a QuantStudio 3 thermocycler (Applied Biosystems) and following the recommendations of the CDC (cdc.gov/coronavirus/2019-ncov/lab/virusrequests.html). The standard curve of the assay was done using the 2019-nCoV_N_Positive Control plasmid (Cat. #10006625, IDT). This plasmid was linearized with ScaI restriction enzyme to improve the dynamic range and assay-to-assay reproducibility of the standard curve. After cleaning the plasmid digestion using the Monarch DNA Cleanup Kit (NEB, #T1030), the DNA concentration of the linearized plasmid was measured to determine the number of DNA molecules in the stock solution, which was calculated using SnapGene software version 5.0.8. Six point concentrations were prepared at 10⁴, 10⁵, 10⁶, 10⁷, 10⁸ and 10⁹ DNA molecules/mL for the standard curve, which was included in every PCR amplification of the antiviral assays. To avoid variability, enough of each point solution was prepared for all the assays performed in this study. Thus, PCR efficiency was consistent between PCR amplifications (between 95 and 105% efficiency).

### Measurement of Infectious Virus by TCID₅₀

ARINA-1-treated or untreated primary fully differentiated HBEC cells were infected with SARS-CoV-2 as described above for the HBEC/ALI-based antiviral assay. After 72 h of incubation the apical side of the cell layers were washed with 100 μl of 180 PBS 1× to collect the virus particles. Collected virus suspensions were serially diluted (5-fold series) in EMEM medium (ATCC, cat. #30-2003™) in sextuplicate and then 30 μL of viral dilution were added to a 30 μL volume of freshly plated Vero E6 cells (ATCC, cat. #CRL-1586™) at 5,000 cells/well in 384-well white plate (Corning, cat. #3570). The plates were incubated at 37° C. under 5% CO₂ for 144 hour and virus cytopathic effect was measured using the CellTiter-Glo® 2.0 Cell Viability Assay (Promega, Cat. #G9241) following the manufacturer instructions. The luminescence values were used to calculate the TCID50 of each virus sample using the Viral ToxGlo™ Assay (promega.com/resources/protocols/technical-manuals/101/viral-toxglo-assay-protocol/).

### Cytotoxicity of Compounds

The cytotoxicity of all compounds (except ARINA-1 that interferes with the assay) was measured using the CytoTox 96® Non-Radioactive Cytotoxicity Assay (Promega, Cat. #G1780), which measures LDH enzyme release. ARINA-1 cytotoxicity was measured using the CellTiter-Glo® 2.0 Cell Viability Assay (Promega, Cat. #G9241), which measures intracellular ATP. All assays were done in 16HBE cells cultured at the ALI in EMEM medium as described above. The cells were exposed to the compounds either basolaterally or apically, depending on their mechanism of action for 72 hours at the highest concentrations used in the antiviral assays. For the LDH kit the basolateral medium was directly assayed following the manufacturer instructions. For the ATP kit, the cells were scraped from the transwell filters and recovered in 100 μl of PBS 1×, transferred to a white opaque 96-well plate, then mixed with 100 μl of CellTiter-Glo reagents, and continued following manufacturer instructions. Toxicity of the compounds were reported as percent of the maximum toxicity of control cells that were treated with a lysis reagent supplied by the kits.

### Assessment of SARS-CoV2 Cytopathic Effect Through Immunohistochemistry

ARINA-1 (having a glutathione: ascorbic acid: bicarbonate molar ratio of about 0.5 0.5:1, pH 6-7) cytoprotective activity of the HBEC was evaluated through immunohistochemistry using cilia and apoptosis markers (acetylated β-tubulin and caspase-3, respectively). The unstained slides of 5 μm paraffin sections of the HBEC were baked overnight at 60° C. then deparaffinized by dissolving with three changes of xylene and hydrated using graded concentrations of ethanol to deionized water. The tissue sections were subjected to antigen retrieval by 0.01 M sodium citrate buffer (pH 6) in a pressure cooker for 5 min (buffer preheated). Following antigen retrieval, all sections were washed gently in deionized water, then transferred into 0.05 M Tris-based solution in 0.15 M NaCl with 0.1% v/v Triton-X-100, pH 7.6 (TBST). Endogenous peroxidase was blocked with 3% hydrogen peroxide for 15 min. To reduce further nonspecific background staining, slides were incubated with 5% normal goat serum (Sigma, G9023) for 45 min at RT. Slides then were incubated at 4° C. overnight with the corresponding primary antibody (anti-acetylated tubulin, Sigma T7451, mouse monoclonal, 1/60,000 dilution; anti-cleaved caspase 3, Cell Signaling 9579, rabbit monoclonal, 1/500 dilution; or anti-SARS-CoV2 glycoprotein S, Invitrogen PA141165, rabbit polyclonal, 1/500 dilution). After washing with TBST, sections corresponding to tubulin staining were incubated with a goat anti-mouse IgG secondary antibody conjugated with HRP (Abcam ab6789, 1:500), while sections of caspase and protein S staining were incubated with goat anti-rabbit IgG secondary antibody conjugated with HRP (Abcam ab6721, 1:1,000). ImmPACT DAB Peroxidase (HRP) Substrate Kit (Vector Laboratories SK4105) was used as the chromogen and hematoxylin (no. 7221, Richard-Allen Scientific, Kalamazoo, MI) as the counterstain. To validate the specificity of the antibodies, control slides were used following the protocol described above, but at the step of primary antibody incubation PBS buffer was used instead of the primary antibody.

### Cilia Length Measurement

Image processing and analysis were implemented using ImageJ v1.53a. An RGB histologic photograph was opened, and each cilium was traced using the draw (pencil) tool from the ciliated columnar epithelial cell surface to the tip of the cilium. After tracing all cilia in the photograph, the data type was converted from RGB to 8 bits, and the cilia images were segmented using a thresholding technique. Then the binary images of cilia were skeletonized, and the total number of cilia and their lengths were measured using the “Analyze Skeleton 2D/3D” function.

### Testing Direct Antiviral Activity of ARINA-1 on SARS-CoV-2 Virion

A 100 μl volume of ARINA-1 or saline was added to 100 μl of SARS-CoV-2 suspension (5.52×10⁸ viral particles/mL) and incubated for 1 hour at 100 μl 37° C. After incubation, the mixtures were added to 3K MWCO 0.5 mL filter (Pierce, Cat. No. 88512) and centrifuged at maximum speed to concentrate the virus 10 times (to a volume of 20 l). Then, 300 μl of PBS 1× were added to the filter, and the virus was concentrated again by centrifugation to 20 μl volume. This PBS wash was repeated two times to eliminate the rest of the ARINA-1 components, and the recovered viral suspension was adjusted to the original volume of 100 μl to keep the same viral concentration as the original suspension. A 30 μl volume of this processed viral suspension was used to infect Vero E6 cells plated at the confluence in a 96 well plate or HBEC in ALI filters. The viral load was measured by qRT-PCR as described above. Controls were run in parallel using saline instead of ARINA-1.

### μOCT Image Acquisition and Analysis.

Measurements of functional microanatomic parameters in cultured primary HBECs were performed using OCT as previously described (Peabody, J. E. et al., American Journal of Physiology—Lung Cellular and Molecular Physiology 314, L909-L921 (2018); Liu, L. et al., PLoS One 8, (2013); and Kaza N, Lin V Y, Stanford D, et al., European Respiratory Journal. Published online Dec. 16, 2021:2101581. doi:10.1183/13993003.01581-2021). Specifically, MCT rate was determined using the time elapsed and distance traveled of native particulates in the mucus over multiple frames. Four videos were randomly acquired at standard distances from the center of the wells (mid-way between the center and the border circle) for each well. MCT rates were normalized against the MCT average of baseline controls (uninfected cells treated with saline).

### Example 2: HBEC/ALI-Based Antiviral Assay Validation

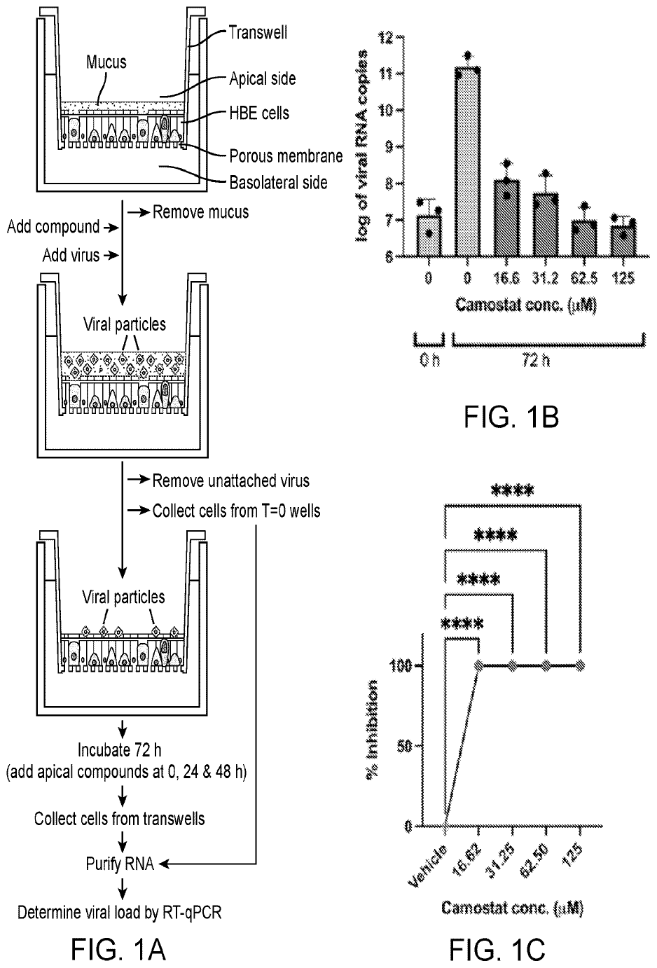

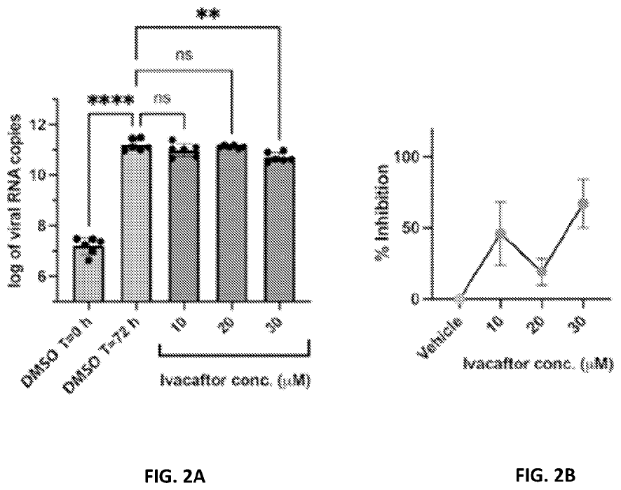

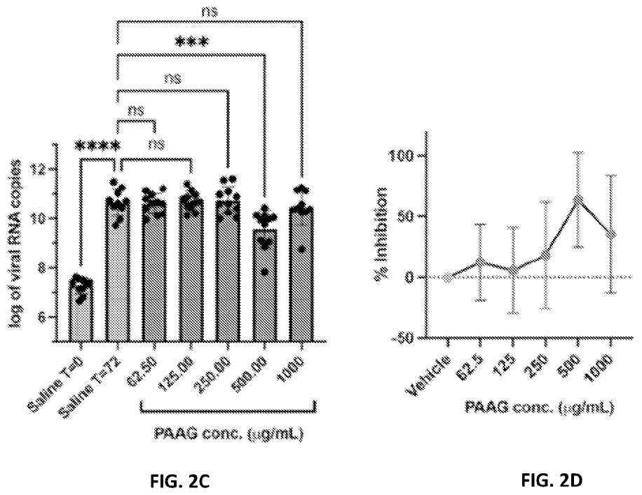

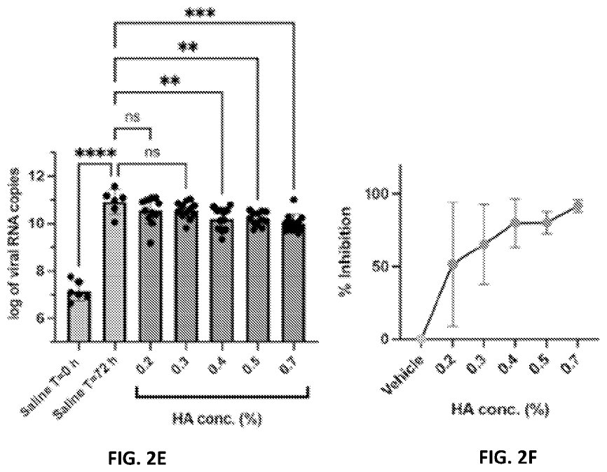

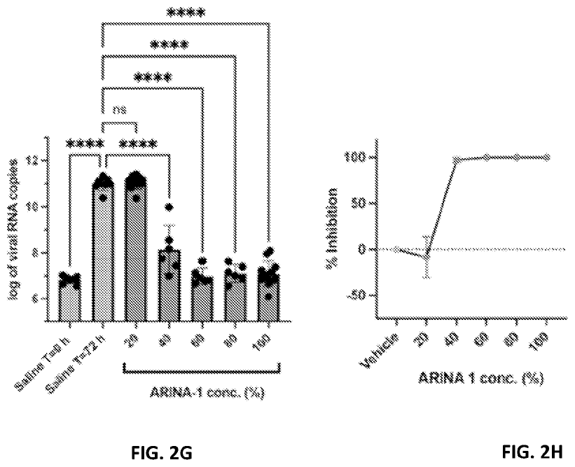

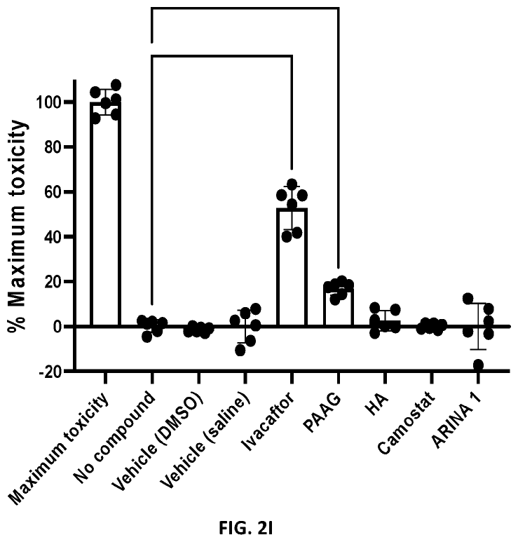

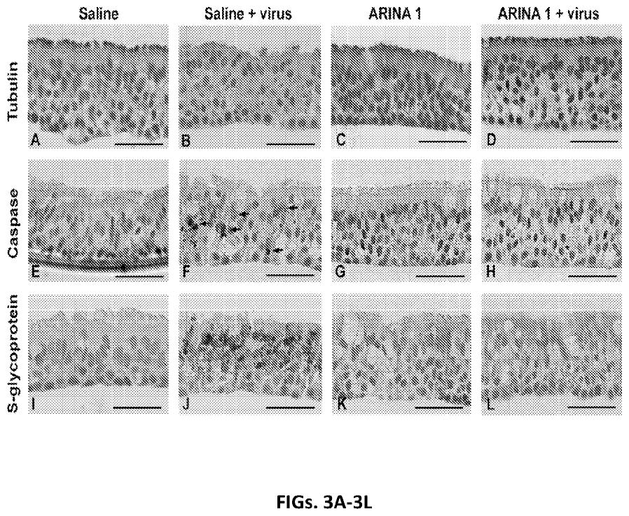

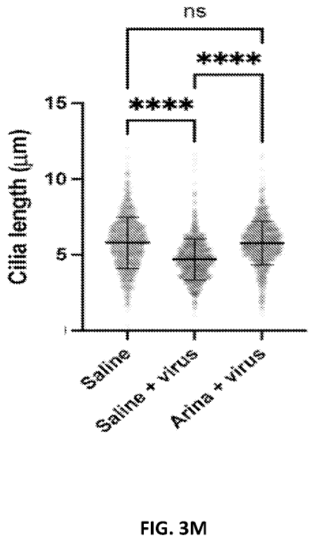

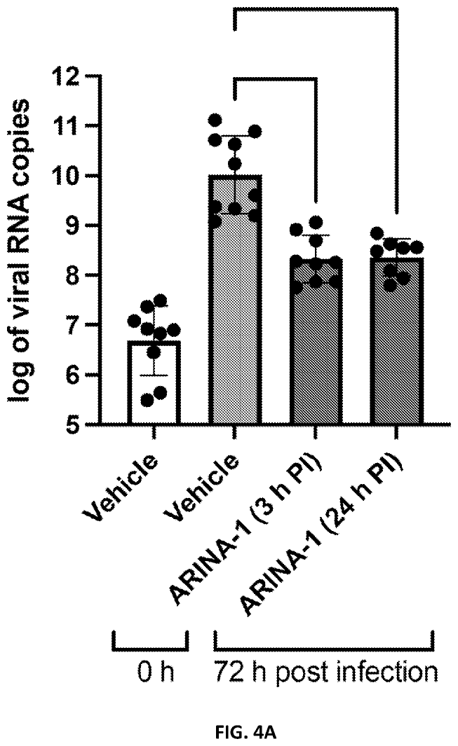

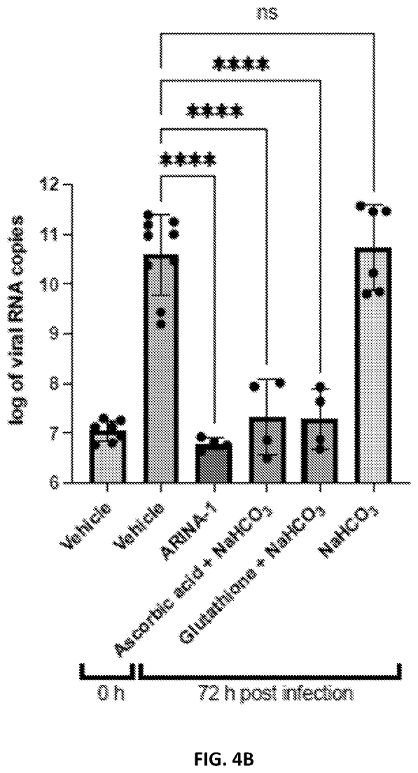

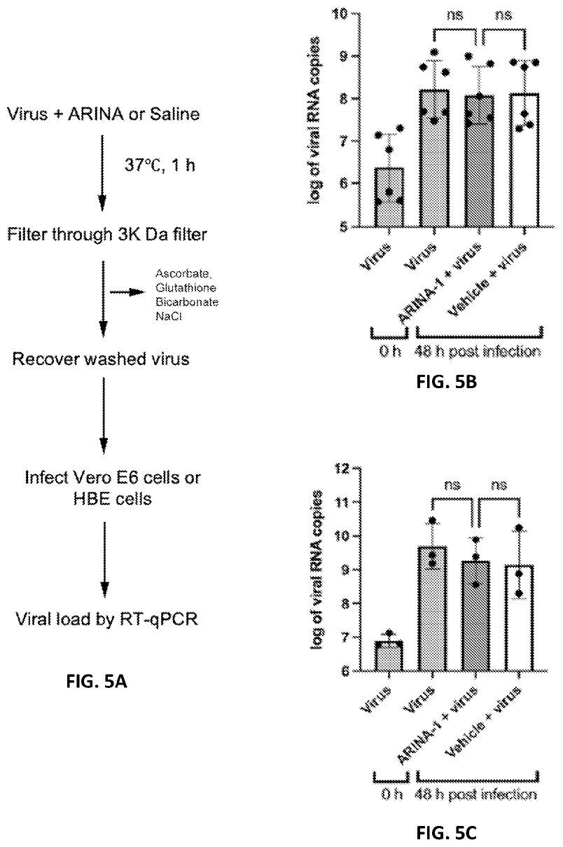

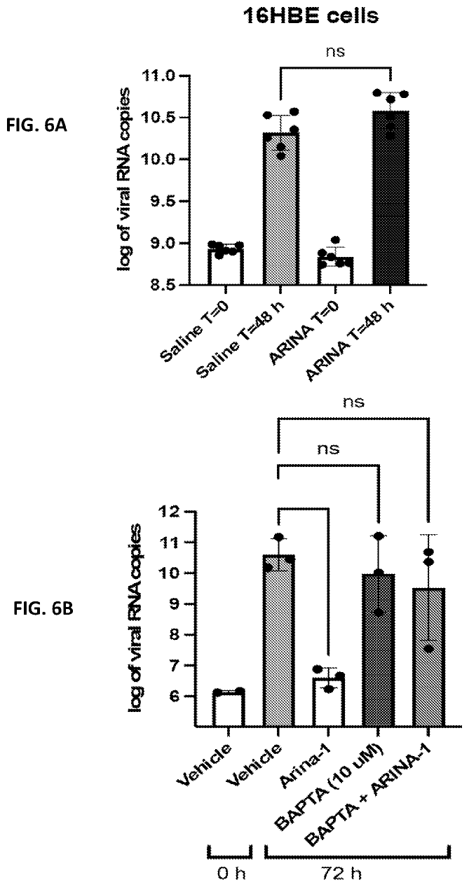

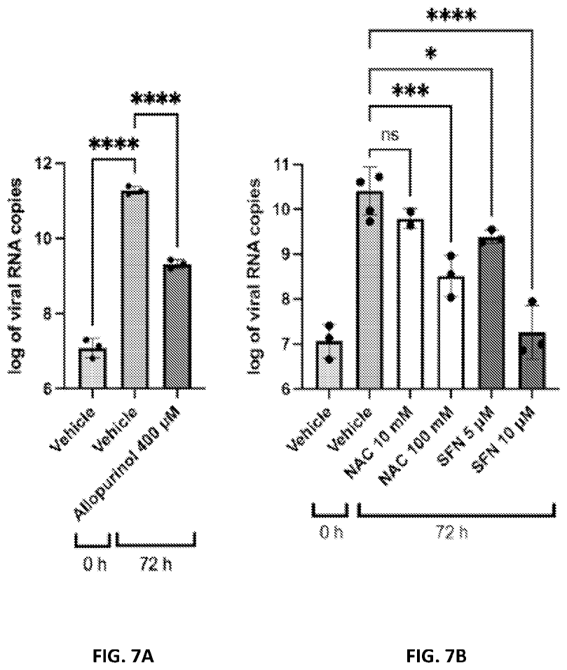

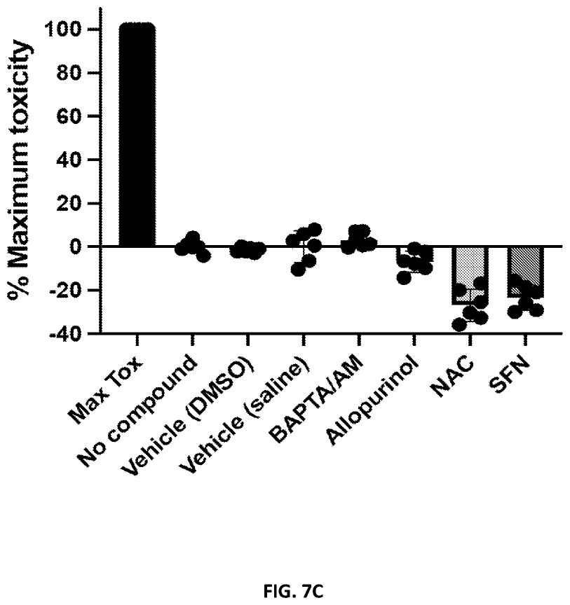

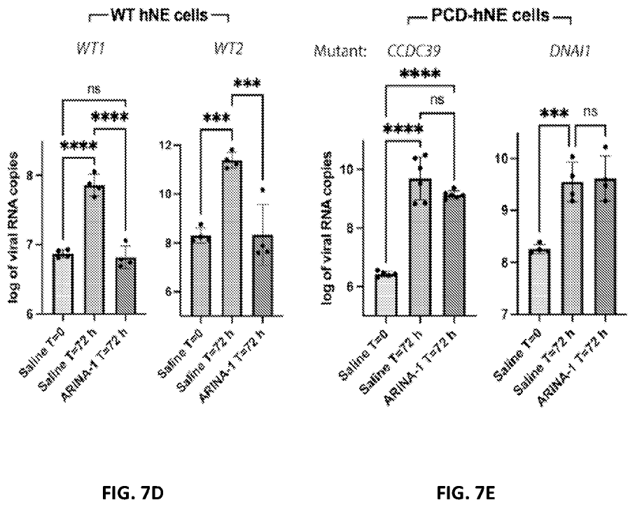

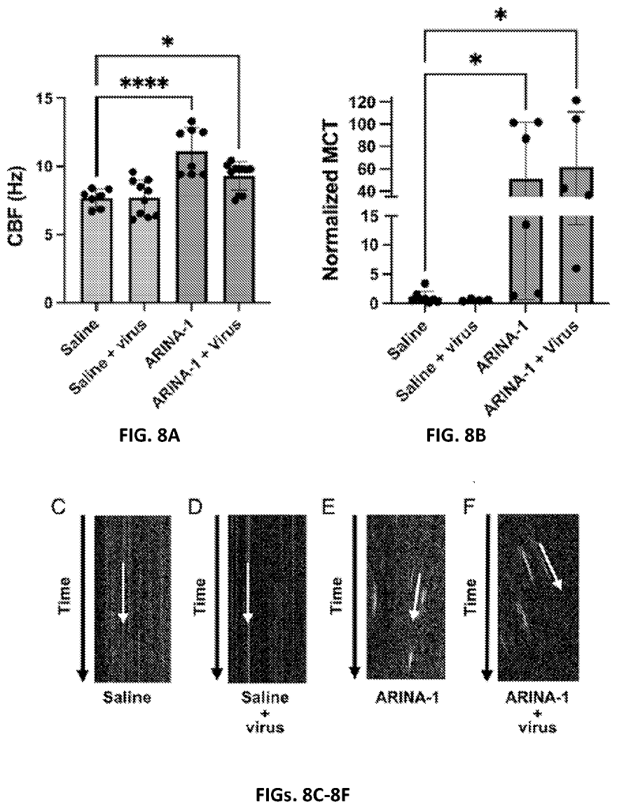

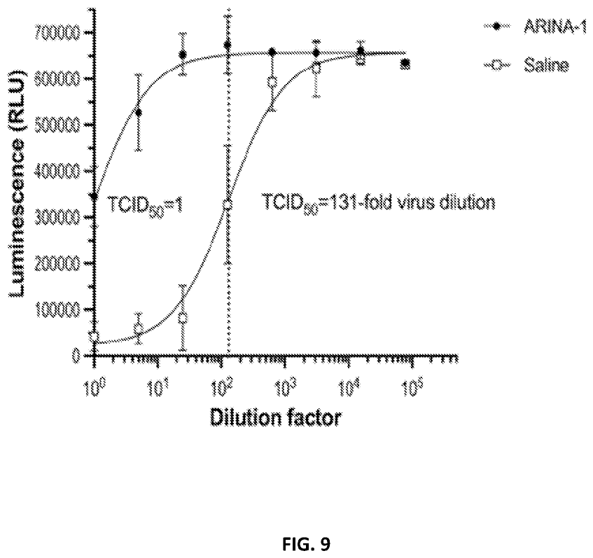

The antiviral assays were performed as described in the Example 1 (a simplified scheme of the assay is shown in FIG. 1A). The assay was validated using camostat mesylate compound due to its dual action in blocking SARS-CoV2 entry to the cell through the inhibition of TMPRSS2-mediated priming of the viral S protein (Hoffmann, M. et al., Cell 181, 271 (2020)) and its mucoactive effect via the inhibition of the epithelium sodium channel (ENaC)-activating protease, which enhances MCT (Coote, K. et al., J Pharmacol Exp Ther 329, 764-774 (2009)). It was found that camostat mesylate inhibited the replication of SARS-CoV2 in HBECs in a dose-dependent manner (FIG. 1i). Camostat mesylate inhibited SARS-CoV2 infection nearly 100% inhibition at concentrations higher than 16.62 μM when compared with the DMSO vehicle (FIG. 1C). The compound was not toxic to the cells at the maximum concentration tested in the antiviral assays (125 μM, FIG. 2), ruling out that the observed decrease in viral load was due to cell death caused by the drug. Camostat mesylate was found to be partially protective against SARS-CoV2 in Vero cells. These results validated the anti-viral assay in experimental conditions.

### Example 3: Effect of Mucoactive Compounds on SARS-CoV2 Infection of HBECs

Five mucociliary active drugs were evaluated for potential antiviral activity: ivacaftor, poly-N(acetyl, arginyl) glucosamine (PAAG), high molecular weight hyaluronic acid (HA), camostat mesylate and ARINA-1. Ivacaftor is a potentiator of the chloride ion transporter cystic fibrosis transmembrane conductance regulator (CFTR), which works by improving the function of the CFTR protein in cystic fibrosis (CF) patients with a gating defect (Kotha, K. & Clancy, J. P., Therapeutic Advances in Respiratory Disease 7, 288-296 (2013); Raju, S. V. et al., American Journal of Respiratory Cell and Molecular Biology 56, 99-108 (2017)). By restoring the flow of chloride ions through the cell membrane via CFTR, ivacaftor improves the MCT and thereby reduces CF symptoms such as the build-up of thick mucus in the lung. As previously shown, it has some bioactivity in wild type epithelia (Raju 2017). The synthetic glycopolymer PAAG improves the viscoelasticity of the airways' mucus, which reduces the characteristic high viscosity of CF patients' mucus and restores the MCT (Fernandez-Petty, C. M. et al., JCI Insight 4, (2019)). The biopolymer HA, particularly its high molecular weight form, is an important constituent of the extracellular matrix of the lungs with anti-inflammatory and water-retaining properties. Thus, HA plays a significant role in the regulation of fluid balance in the lung and airway interstitium and has a favorable effect on MCT (Zahm, J. M., Milliot, M., Bresin, A., Coraux, C. & Birembaut, P., Matrix Biology 30, 389-395 (2011); Johnson, C. G. et al., American Journal of Physiology—Lung Cellular and Molecular Physiology 315, L787-L798 (2018); Miiz Carro, L. & Martinez-Garcia, M. A., Cells 9, (2020); and Galdi F, Pedone C, McGee C A, et al., Respiratory Research. 2021; 22(1):1-11). Camostat mesylate has is mucoactive via the inhibition of the epithelium sodium channel (ENaC)-activating protease, which results in MCT enhancement. In addition, camostat mesylate is known to block SARS-CoV2 entry to the cell through the inhibition of TMPRSS2 protease-mediated priming of the viral S glycoprotein. Finally, ARINA-1, a nebulized formulation was tested for potential antiviral activity. Our rationale for testing Camostat and ivacaftor basolaterally was that this approximates their use as systemic agents. The other compounds tested (PAAG, HA and ARINA-1) are hydrophilic, were used in very low volumes (1 ul) and have been used previously directly on the apical side of airway epithelia. This also mimics their use as inhaled agents.

In the antiviral assays, all tested compounds showed some degree of inhibition of SARS-CoV-2 replication in the HBECs. Accordingly, these compounds and related compounds (e.g., pharmaceutically acceptable salts thereof, derivatives, conjugates) are expected to be inhibitory for SARS-CoV-2 replication in HBECs. Ivacaftor was administered basolaterally into the medium and, had a moderate effect on SARS-CoV-2 replication at concentrations close to its maximum solubility (≥10 μM) (FIGS. 2A-2B). PAAG also showed a moderate effect, with a peak of antiviral activity at 500 μg/ml (1 ul, applied apically) that decreased at higher concentration (FIGS. 2C-2D). However, both compounds showed cytotoxicity (FIG. 2I), which makes it difficult to discern what portion of the observed reduced viral load is due to its antiviral activity or direct cytotoxicity.

However, higher antiviral activity was observed with HA, reaching more than 90% inhibition at a concentration of 0.7% (1 μl, apical) (FIGS. 2E-2F). The individual components either alone or buffered have no antiviral or MCT activity. The fixed dose combination of ARINA-1 exhibited the most potent antiviral activity, achieving an unexpected greater than 99% inhibition at concentrations 40% and higher of the original formulation (FIGS. 2G-2H). Neither HA nor ARINA-1 showed cytotoxicity at their tested maximum concentrations (0.7% and 100%, respectively). Particularly, high-molecular-weight HA has been evaluated in clinical trials for its potential protection against progression of COVID-19-induced respiratory failure; however, the outcomes of this study have not yet been published (Use of Inhaled High-molecular Weight Hyaluronan in Patients With Severe COVID19: Feasibility and Outcomes—Full Text View—ClinicalTrials.gov [Online]. clinicaltrials.gov/ct2/show/NCT04830020). On the other hand, ARINA-1 has not been evaluated for its potential benefits in COVID-19 disease. Furthermore, given ARINA-1's established strong safety profile in chronic toxicology studies and its emerging clinical safety profile, further studies on ARINA-1 were conducted to assess ARINA-1 as a mucoactive agent with potential anti-SARS-CoV-2 activity in respiratory epithelia and mechanistic link to ciliary function.

### ARINA-1 Inhibits the Shedding of Infectious Virus by Primary HBEC

To ascertain that ARINA-1-caused reduction of SARS-CoV-2 RNA amounts (measured by RT-qPCR) correlated with the blocking of viral replication, we quantified the amount of infectious virus shed by the primary HBEC cells using a median tissue culture infection dose (TCID₅₀) determination assay. For this, we washed the apical side of the HBEC to collect virus from ARINA-1-treated or untreated samples and used serial dilutions of the recovered virus to infect Vero E6 cells for its quantification. Thus, we compared the amounts of virus shed by ARINA-1-treated primary HBEC with the mock treated cells (saline) and showed that ARINA-1 blocked the production of infectious virus by two orders of magnitude in HBEC when compared to saline-treated controls (TCID₅₀=1 vs 131-fold dilution, respectively, FIG. 9). This result indicated that the amounts of viral RNA measured by RT-qPCR correlated with the amounts of infectious virus in our HBEC/ALI-based antiviral assay and further validated it as a method for the search of anti-SARS-CoV-2 drugs.

### Example 4: ARINA-1 Protects HBECs from SARS-CoV2-Driven Cytopathogenicity

It has been well documented that SARS-CoV2 causes extensive plaque-like cytopathic effects in HBEC cultures, including cell fusion, apoptosis, destruction of epithelium integrity, cilium shrinkage, and granular formation on cilia. In the antiviral assay disclosed herein, the HBECs treated with the vehicle (saline) showed the characteristic cytopathic effects caused by SARS-CoV2. However, the cells treated with ARINA-1 were protected from the cytopathic effects caused by SARS-CoV2. Specifically, it was observed that ARINA-1 protected the ciliated cells from cilia shrinkage and loss, as well as from the SARS-CoV2-induced apoptosis (FIGS. 3A-3L). In addition, the sections of the mock-treated epithelial layer that showed more cell damage and apoptosis corresponded with higher infection, as demonstrated by higher immunostaining for the specific viral marker S glycoprotein of SARS-CoV2 (FIGS. 3A-3L). However, ARINA-1-treated cells did not show any specific S glycoprotein immunostaining, which strongly indicated that cells were protected from SARS-CoV2 infection (FIGS. 3A-3L), suggesting augmented MCT was diminishing cell entry and that ARINA-1 may have direct protective mechanisms on the airway epithelial cells to prevent injury and apoptotic signaling.

### Example 5: ARINA-1 Blocks SARS-CoV2 Replication Even when Administered after Viral Infection

Given that ARINA-1 significantly prevents SARS-CoV2 infection of HBECs, a test was performed to determine if the treatment of already infected cells could decrease the spread of infection and ameliorate the viral load. For this, HBECs were infected for 1 hour and then washed off excess viral particles, limiting infection of the epithelial cells to endogenous virus. Cells were then treated with ARINA-1 at 3 and 24 hours after the infection onset and assessed viral load at 72 hours after exposure to the virus. It was observed that cells treated with ARINA-1 at both 3 and 24 hours post-infection significantly reduced the viral load 72 hours after exposure to the virus (FIG. 4A). Because ARINA-1 reduced viral load when added to the HBE cells after 24 h of virus exposure (FIG. 4A), ARINA-1 can be used as a therapeutic for subjects already infected with SARS-CoV-2 or similar viruses, as the beneficial effect on viral replication could be conferred through diminishing cell-to-cell spread through apical transmission, as well as decreasing or limiting damage to the airway epithelial cells.

### Example 6: Determining the Active Components of ARINA-1

To evaluate the overall effect of ARINA-1 as well as the contribution of each of these components to the observed antiviral activity, the antiviral activity of glutathione plus bicarbonate, ascorbic acid plus bicarbonate, and bicarbonate alone were tested, each at the same concentrations in ARINA-1. Bicarbonate was required to buffer glutathione and ascorbic acid because glutathione ascorbic acid alone have been shown to be cytotoxic due to the very low pH of the compounds. The results show that both glutathione plus bicarbonate and ascorbic acid plus bicarbonate each have a significant antiviral effect. However, bicarbonate alone did not show significant activity (FIG. 4B).

These results demonstrated that the active antiviral compounds in ARINA-1 are the antioxidant agents glutathione and ascorbic acid, and our results demonstrate that bicarbonate may not contribute to the antiviral activity when it is mixed with each antioxidant separately. However, a contribution by bicarbonate, beyond its acid buffering capacity such as its indirect influence on MCT (Impaired mucus detachment disrupts mucociliary transport in a piglet model of cystic fibrosis—PubMed [Online]. pubmed.ncbi.nlm.nih.gov/25124441/; Birket S E, et al., Am J Respir Crit Care Med 190: 421-432, 2014. doi: 10.1164/RCCM.201404-06700C.) was not fully discarded because glutathione and ascorbic acid was not tested without bicarbonate.

### Example 7: ARINA-1 is not Directly Deleterious to SARS-CoV2

In addition to testing if mucoactive agents can protect against SARS-COV2 by augmenting MCT, the direct anti-viral activity of ARINA-1 on SARS-CoV2 virus was evaluated. A suspension of SARS-CoV2 virus was exposed to ARINA-1 and then ARINA-1 was removed by ultrafiltration using a pore size that allows only ARINA-1 to be removed but not the virus particles (FIG. 5A). It was found that the virus particles exposed to ARINA-1 were as infective as those exposed to saline solution in Vero E6 cells (FIG. 5B) and in HBEC grown in an ALI (FIG. 5C). These data demonstrate that the unexpected replication inhibition effects of ARINA-1 (described above) are not a result of direct damage to SARS-CoV2 virions, rather the MCT mechanism of ARINA-1.

### Example 8: MCT Accounts for Most of the Anti-SARS-CoV2 Protection Conferred by ARINA-1

Given the efficacy of ARINA-1 in blocking SARS-CoV2 infection, it was further investigated if its antiviral activity can be explained by the improvement on MCT alone or if there may be another ARINA-1-triggered cellular mechanism of protection involved or interference with the viral and post-entry pathways. To answer this question, antiviral assays in which MCT was not present or was blocked and evaluated if ARINA-1 could still confer total or partial protection were devised. For the first approach in which the MCT not present, undifferentiated 16HBE cells (immortalized 16HBE14o- cells) were used, and primary HBECs were grown in an ALI for only one week, before mucociliary differentiation is achieved, so that neither cell line had cilia present and MCT was therefore absent (Garcii S R, et al., Development 146, 2019. doi: 10.1242/DEV.177428; Cozens A L, et al., Am J Respir Cell Mol Biol 10: 38-47, 1994. doi: 10.1165/AJRCMB.10.1.7507342). The undifferentiated 16HBE cells were treated with ARINA-1 or saline and infected (FIG. 6A). Under these conditions, ARINA-1 was unable to inhibit SARS-COV2 infection, without being bound by theory, these results suggest MCT may be needed to exhibit its inhibitory properties or SARS-CoV-2 replication. In this study, the impact of ARINA-1 on apoptosis was not evaluated.

In the second set of experiments, MCT was blocked using a BAPTA-AM, an inhibitor of cilia motility. BAPTA-AM is a membrane permeable selective calcium chelator. Since calcium is required for cilia motility, sequestering of this important ion by BAPTA-AM immobilizes the cilium beating. In the presence of BAPTA-AM (10 uM), ARINA-1 did not show antiviral activity (FIG. 6B). The compound was not cytotoxic at the concentration used (FIG. 7C). Taken together, these results suggest that cilia motility is required for ARINA-1-dependent antiviral protection.

ARINA-1 was also evaluated in human nasal epithelial cells (hNEs) from two patients affected by primary ciliary dyskinesia (PCD) syndrome, a genetic condition characterized by defective cilia expression or function (Horani A, et al., Paediatr Respir Rev 18: 18, 2016. doi: 10.1016/J.PRRV.2015.09.001). Similar to the HBEC, hNE cells from healthy donors (WT-hNE cells) and from the PCD donors (PCD-hNE cells) were terminally differentiated at ALI, and then the effect on SARS-CoV-2 replication assessed. ARINA-1 did not protect against SARS-CoV-2 infection in the PCD-hNE cells; however, it remained inhibitory in the WT-hNE cells (FIGS. 7D and 7E, respectively). Taken together, these results indicate that ciliary motility is required for ARINA-1-dependent antiviral activity and that MCT is the primary mechanism of action of the antiviral effect of ARINA-1 seen in the ALI-primary HBEC infection model.

### Example 9: The Oxidative State of the Cells Contributes Antiviral Protection

Proper cilia functioning requires a balanced redox environment, which is maintained by redox regulatory proteins and thioredoxin domain-containing proteins (Price, M. E. & Sisson, J. H., Redox Biology 27, 101146 (2019)). Dynein ATPases, which are the ciliary protein motors, as well as several cilia-localized proteins that modulate dynein activity (e.g. Protein Kinase A, Protein Kinase C, and Protein Phosphatase 1), are sensitive to the local redox environment within each cilium. As noted, the active antiviral compounds of ARINA-1 are ascorbic acid and glutathione which are known for their antioxidant capacity, and formulated with bicarbonate as a buffering agent. Whether the antioxidant aspect of the molecules is responsible for the improvement of the MCT and consequent antiviral activity was investigated. It was hypothesized that reducing the endogenous production of reactive oxygen species (ROS) by HBECs can simulate the anti-SARS-CoV2 protection conferred by ARINA-1. To reduce endogenous ROS production, xanthine oxidase was inhibited. Xanthine oxidase catalyzes the oxidation of hypoxanthine to xanthine and can further catalyze the oxidation of xanthine to uric acid, generating ROS in the process. Hypoxanthine is derived from the catabolism of ATP via AMP. Thus, energy-demanding activities that consume ATP, such as cilia beating, or muscle contraction generate ROS through the xanthine oxidase pathway (Heunks, L. M. A. et al., American Journal of Physiology—Regulatory Integrative and Comparative Physiology 277, (1999); VinÑa, J. et al., IUBMB Life 50, 271-277 (2000); and Vina, J. et al., IUBMB Life 49, 539-544 (2000)). It was observed that inhibition of xanthine oxidase with allopurinol significantly protected the HBECs from SARS-CoV2 infection (FIG. 7A).

In addition, whether antioxidants other than glutathione and ascorbic acid could also confer antiviral protection was tested. This was observed by testing N-Acetyl-L-cysteine (NAC) and sulforaphane (SFN). Both compounds caused a reduction in the viral load when tested in the HBEC/ALI antiviral assay (FIG. 7B), and neither allopurinol, NAC nor SFN showed cytotoxicity at the higher concentrations tested (400 μM, 100 mM, and 10 μM, respectively (FIG. 7C)). These results suggest that antioxidants may benefit epithelial cell resistance to SARS-CoV-2 by imposing a redox state favorable to MCT and may explain, at least in part, the beneficial effects of ARINA-1.

### Example 10: ARINA-1 Induces a Supernormal MCT and Hyper Ciliation in HBECs

Additionally, how ARINA-1 affected MCT in uninfected and SARS-CoV2-infected HBECs was tested using micro-optical coherence tomography (μOCT). This allowed the cross-sectional study of the live HBEC surface at a microscopic level. Cilia beat frequence was measured according previously described methods (Li, W. E. et al., Pflugers Archiv: European journal of physiology 464, 671-680 (2012)). Measurement of cilia beating frequency (CBF) by OCT showed that in ARINA-1-treated cells, even after infection with SARS-CoV-2, cilia CBF was significantly higher than in the saline-treated controls (FIG. 8A). Correspondingly, MCT was significantly augmented in ARINA-1-treated cells compared to those treated with saline (FIG. 8B), even in SARS-CoV-2 infected cells compared to mock controls (FIGS. 8C to 8F), of SARS-CoV-2 on mucociliary function. It was observed that ARINA-1 significantly increased MCT compared to mock-treated cells (FIG. 8A). However, no significant difference was observed between uninfected and infected HBEC that were treated with ARINA-1, although in both cases the MCT was elevated compared to their respective mock-treated controls (FIG. 8A).

Taken together, these results disclosed herein indicate that ARINA-1-mediated improvement of MCT is the main contributor to antiviral protection in the HBEC/ALI model of study employed.

The reduction in cilia length, i.e., cilia shrinkage, observed in histology preparations was quantified following the algorithm described as follows. Image processing and analysis were implemented using ImageJ v1.53a. An RGB histologic photograph was opened, and each cilium was traced using the draw (pencil) tool from the ciliated columnar epithelial cell surface to the tip of the cilium. After tracing all cilia in the photograph, the data type was converted from RGB to 8 bits, and the cilia images were segmented using a thresholding technique. Then the binary images of cilia were skeletonized, and the total number of cilia and their lengths were measured using the “Analyze Skeleton 2D/3D” function. A significant reduction in cilia length was observed in SARS-CoV-2-infected compared to uninfected HBECs (FIG. 3M). In contrast, when ARINA-1 was administered 1 hour prior to infecting the cells, cilia shrinkage was not observed (FIG. 3M). Together, these results indicated beneficial effects on ciliary structure and function that imparted improved MCT.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

What is claimed is:

1. A method of treating, preventing, or reducing the risk of an infection by SARS-CoV-2 in a subject in need thereof, comprising administering to the subject a therapeutically effective amount of a composition comprising:
(a) glutathione, a glutathione derivative, a glutathione conjugate, or a pharmaceutically acceptable salt of glutathione, a glutathione derivative, or a glutathione conjugate; and
(b) an organic acid or a pharmaceutically acceptable salt thereof.

2. A method of reducing one or more symptoms or signs of an infection by SARS-CoV-2, comprising administering to the subject a therapeutically effective amount of a composition comprising:
(a) glutathione, a glutathione derivative, a glutathione conjugate, or a pharmaceutically acceptable salt of glutathione, a glutathione derivative, or a glutathione conjugate; and
(b) an organic acid or a pharmaceutically acceptable salt thereof.

3. The method of claim 2, wherein the one or more symptoms or signs of infection by SARS-CoV-2 comprise lung inflammation, fever, chills, cough, shortness of breath, difficulty breathing, fatigue, muscle aches, body aches, headache, reduction in ability to taste, reduction in ability to smell, sore throat, congestion, runny nose, nausea, vomiting, diarrhea, chest pain, confusion, inability to wake, inability to stay awake, discolored skin, discolored lips, or discolored nail beds.

4. A method of protecting the lungs of a subject suffering from SARS-CoV-2 from cilia damage comprising administering to the airway of the subject a composition comprising:
(a) glutathione, a glutathione derivative, a glutathione conjugate, or a pharmaceutically acceptable salt of glutathione, a glutathione derivative, or a glutathione conjugate; and (b) an organic acid or a pharmaceutically acceptable salt thereof.

5. The method of claim 4, wherein the cilia damage is cilia shortening.

6. The method of any one of claims 1-5, wherein the molar ratio of (a):(b) is about 0.1-0.5:0.5-1.

7. The method of any one of claims 1-6, wherein the composition further comprises (c) bicarbonate or a pharmaceutically acceptable salt thereof.

8. The method of claim 7, wherein the composition comprises sodium bicarbonate or calcium bicarbonate.

9. The method of claim 7 or claim 8, wherein the molar ratio of (a):(b):(c) is about 0.1-0.6:0.5-1:1, about 0.4-0.6:0.4-0.6:1, or about 0.5:0.5:1.

10. The method of any one of claims 1-9, wherein SARS-CoV-2 is a variant is selected from the group consisting of an Alpha variant (e.g., B.1.1.7 and Q lineages), a Beta variant (e.g., B.1.351 and descendent lineages), a Gamma variant (e.g., P.1 and descendent lineages), a Delta variant (e.g., B.1.617.2 and AY lineages), an Epsilon variant (e.g., B.1.427 and B.1.429), an Eta variant (e.g., B.1.525), an Iota variant (e.g., B.1.526), a Kappa variant (e.g., B.1.617.1), 1.617.3, a Mu variant (e.g., B.1.621, B.1.621.1), a Zeta variant (e.g., P.2), or an Omicron variant (e.g., B.1.1.529, BA.1, BA.1.1, BA.2, BA.3, BA.4 and BA.5 lineages).

11. The method of any one of claims 1-10, wherein the composition comprises glutathione.

12. The method of any one of claims 1-11, wherein the organic acid is ascorbic acid or a pharmaceutically acceptable salt thereof.

13. The method of any one of claims 1-12, wherein the composition is administered to the lungs by an inhalable dosage form.

14. The method of claim 13, wherein the inhalable dosage form is a metered dose inhaler, a dry powder inhaler, or a nebulizer.

15. The method of any one of claims 1-14, wherein the subject has long COVID.

16. The method of any one of claims 1-15, wherein the subject has a chronic airway disease or condition.

17. The method of any one of claims 1-16, wherein the subject belongs to a subject population having an increased risk of SARS-CoV-2 infection, or an increased risk of a severe infection by SARS-CoV-2.

18. The method of any one of claims 1-17, wherein the subject is aged 65 or greater.

19. The method of any one of claims 1-18, wherein the subject suffers from an acute SARS-CoV-2 infection.

20. The method of any one of claims 1-19, wherein the subject has developed one or more SARS-CoV-2-related sequelae.

21. The method of claim 20, wherein the one or more SARS-CoV-2-related sequelae comprises a bronchiectasis.

22. The method of claim 21, wherein the bronchiectasis is traction bronchiectasis.

23. The method of claim 21, wherein the bronchiectasis is non-cystic fibrosis bronchiectasis.

24. The method of claim 20, wherein the one or more SARS-CoV-2-related sequelae is characterized by a symptom selected from one or more of ageusia, myalgia, arthralgia, parosmia, anosmia, fatigue, headache, cough, chills, shivers, fever, dyspnea, sore throat, rhinorrhea, diarrhea, brain fog, nausea, subjective fever, abdominal pain, vomiting, rash, skin abnormality, and blood clots.

25. The method of any one of claims 1-24, wherein the composition blocks SARS-CoV-2 replication in the cells of the subject's airway.

26. The method of any one of claims 1-25, wherein the composition reduces SARS-CoV-2 viral load in the cells of the subject's airway.

27. The method of any one of claims 1-26, wherein the composition does not decrease SARS-CoV-2 infectivity in the cells of the subject's airway.

28. The method of any one of claims 1-27, wherein the method (i) increases the ciliary beat frequency of the patient's airway epithelial cells, (ii) increases the number of the cilia in the airway of the subject, and/or (iii) increases the length of the cilia in the airway of the subject.
